(19) United States
(12) Patent Application Publication
(10) Pub. No.: US 2020/0002724 A1
(43) Pub. Date: Jan. 2, 2020

(54) PACS1 ENHANCEMENT FOR REV-DEPENDENT LENTIVIRAL VECTORS

(71) Applicant: Baylor College of Medicine, Houston, TX (US)

(72) Inventors: Andrew Patrick Rice, Houston, TX (US); Sona Budhiraja, Houston, TX (US); Hongbing Liu, Houston, TX (US); Pei-Wen Hu, Houston, TX (US)

(21) Appl. No.: 16/489,187

(22) PCT Filed: Feb. 26, 2018
(86) PCT No.: PCT/US18/19726

§ 371 Date: Aug. 27, 2019

Related U.S. Application Data
(60) Provisional application No. 62/465,420, filed on Mar. 1, 2017.

(51) Int. Cl. C12N 15/86; C12N 7/00; C07K 14/47; A61K 48/00

(52) U.S. Cl. CPC C12N 15/86; C12N 7/00; C12N 2740/15052; A61K 48/0066; C12N 2740/15043; C07K 14/4702

(57) ABSTRACT

Embodiments of the disclosure concern systems, methods and compositions for generating Rev-dependent lentiviral vectors. In specific embodiments, the vectors are generated in a system that comprises Phosphofurin Acid Cluster Sorting protein 1 (PACS1) and/or Phosphofurin Acid Cluster Sorting protein 2 (PACS2). In embodiments, the vectors are utilized for therapeutic or research applications.

This application claims priority to U.S. Provisional Patent Application Ser. No. 62/465,420, filed Mar. 1, 2017, which is incorporated by reference herein in its entirety.

### STATEMENT REGARDING FEDERALLY SPONSORED RESEARCH OR DEVELOPMENT

This invention was made with government support under AI R21AI114335 awarded by National Institutes of Health. The government has certain rights in the invention.

### TECHNICAL FIELD

Embodiments of the present disclosure include at least the fields of research, therapy, vector technology, virology, gene therapy, and nucleic acid recombination.

### BACKGROUND

The HIV-1 replication cycle is dependent upon cellular co-factors to mediate the various steps in the viral life cycle. A meta-analysis of co-factors reported in the literature and those identified in genome-wide screens concluded that over 2,000 cellular proteins likely have a role in the HIV-1 replication cycle (Bushman, el al., 2005). The identification of co-factors and their mechanisms of action can provide broad insight into both HIV-1 and key cellular processes that are targeted by the virus. This is especially true for the HIV-1 Rev protein—studies of Rev and its co-factors have provided important insight into export of RNA from the nucleus to the cytoplasm.

Rev activates the nuclear export of incompletely spliced viral RNAs. To achieve this, Rev contains an RNA-binding domain that binds to a structured RNA element, the Rev-Response Element (RRE), present in unspliced and incompletely spliced viral transcripts. Rev also contains a nuclear export signal that binds to a nuclear export factor termed CRMI (XPO1) (reviewed in Shida, 2012 and Fernandes, 2016). The Rev-CRtM1-HIV-1 RNA complex, along with the co-factor Ran-GTP, accesses an export pathway used by cellular proteins, rRNA, snRNAs, and a subset of cellular mRNAs (Sloan, et al., 2016). As well as directing nuclear export of unspliced viral RNAs, Rev has been shown to affect additional aspects of the HIV-1 life cycle. Rev promotes the translation of RRE-containing mRNAs (Arrigo, 1991, D'Agostino, et al., 1992; Kimura, et al., 1996) and enhances packaging of HIV-1 RNA into virions (Blissenbach, et at., 2010; Brandt, et al., 2007). However, mechanisms involved in the effects of Rev on translation and RNA packaging are largely unknown.

In addition to CRM1 and Ran-GTP, other Rev co-factors have been identified, including DDX3, DDX1, RNA helicase A, HMI, and Matrin 3 (Fang, et al., 2004; Yedavalli, et al., 2004; Li, et al., 1999; Yedavalli, et al., 2010; Yedavalli, et al., 2011; Kula, et al., 2011). A human nuclear complexome database—the set of protein complexes in the nucleus of HeLa cells (Malovannaya, et al., 2011)—was recently mined that identified RBM14 as a CRM1-associated protein that functions as a Rev co-factor (Budhiraja, et al., 2015). RBM14 is a component of nuclear structures called paraspeckles which are implicated in Rev function (Zhang, et al., 2013)

In analysis of the human nuclear complexome, PACS1 (Phosphofbrin Acid Cluster Sorting protein 1) was identified as a CRM1-associated protein. SiRNA depletion of PACS1 reduced Rev activation of a reporter plasmid, suggesting that PACS1 is a Rev co-factor. PACS1 has previously been identified as a cellular factor of importance to HIV-1 replication. PACS1 mediates localization of Furin to the trans-Golgi network (TCN); Furin is a protease that cleaves the viral gp160 Envelope protein into gp41 and gp120 (Wan, et al., 1998; Hallenberger, et al., 1992). Additionally, PACS1 binds to the viral Nef protein and is involved in down-regulation of MHC I during infection (Piquet, et al., 2000; Blagoveshchenskaya, et al., 2002; Dikeakos, et al., 2012). Although PACS1 is predominantly a cytoplasmic protein (Dikeakos, et at., 2012), a recent study reported that a PACS1-GFP fusion protein accumulates in the nucleus when the CRM1 nuclear export pathway is inhibited with Leptomycin B (Atkins, et al., 2014). This observation indicates that PACS1 shuttles between the nucleus and cytoplasm, a property consistent with a role as a Rev co-factor.

The present disclosure provides solutions to the need in the art for improved production of lentiviral vectors.

### BRIEF SUMMARY

In particular embodiments of the disclosure, there are compositions and methods that allow for an increase in the amounts and/or infectivity of Rev-dependent lentiviral vectors. In specific embodiments, increased production of the vectors occurs from the over-expression of part or all of PACS1 and/or PACS2, such as in polynucleotide form.

In particular embodiments, there is a system for producing a lentiviral expression vector, comprising a vector that encodes Phosphofurin Acid Cluster Sorting protein 1 (PACS1) and/or Phosphofurin Acid Cluster Sorting protein 2 (PACS2), In specific embodiments, the lentiviral expression vector is Rev-dependent. The vector that encodes PACS1 and/or PACS2 may or may not encode one or more lentiviral expression vector components. The vector that encodes .PACS 1 and/or PACS2 may or may not be a transfer vector for production of the Rev-dependent lentiviral expression vector. The vector that encodes PACS1 and/or PACS2 may or may not be a packaging vector for production of the Rev-dependent lentiviral expression vector. The vector that encodes PACS1 and/or PACS2 may or may not be a packaging vector for production of the Rev-dependent lentiviral expression vector. The vector that encodes PACS1 and/or PACS2 may or may not be an envelope vector for production of the Rev-dependent lentiviral expression vector. The vector that encodes PACS1 and/or PACS2 may or may not be an envelope vector for production of the Rev-dependent lentiviral expression vector.

In specific embodiments, the vector that encodes PACS1 and/or PACS2 is a plasmid. In specific cases, one or more regulatory regions for expression of the PACS1 and/or PACS2 are inducible or are constitutive, In specific embodiments, one or more regulatory regions for expression of the PACS1 and/or PACS2 are tissue-specific.

The system may be comprised in or be configured to function in a cell, such as a eukaryotic cell.

In specific cases, PACS1 comprises SEQ ID NO:2 or a functionally active derivative of fragment thereof. PACS1 may be encoded by a polynucleotide comprising SEQ ID NO:1 or a functionally active derivative of fragment thereof. PACS2 may comprise SEQ ID NO:4 or a functionally active derivative of fragment thereof. PACS2 may be encoded by a polynucleotide comprising SEQ ID NO:3 or a functionally active derivative of fragment thereof.

In particular embodiments, the system is housed in a cell that overexpresses PACS1 and/or PACS2.

In one embodiment, there is a method of producing a Rev-dependent lentiviral expression vector, comprising the step of exposing a system that produces a Rev-dependent lentiviral expression vector to a vector that encodes Phosphofurin Acid Cluster Sorting protein 1 (PACS1) and/or Phosphofurin Acid Cluster Sorting protein 2 (PACS2), or two vectors each that encode one of PACS1 and PACS2, wherein the system that produces a Rev-dependent lentiviral expression vector comprises at least a transfer vector, one or more packing vectors, and an envelope vector, wherein the method occurs in a eukaryotic cell. In at least some cases, an envelope protein encoded by the envelope vector is not an HIV envelope protein. The envelope protein may be a Vesicular stomatitis virus (VSV-G) envelope protein or Zika virus envelope protein, for example.

In one embodiment, there is a method of transducing a target cell, comprising the step of exposing the target cell to a Rev-dependent lentiviral expression vector, wherein the Rev-dependent lentiviral expression vector was produced from a system that comprised a vector that encoded PACS1 and/or PACS2. In specific embodiments, the target cell is a human cell, for example a diseased cell. In specific embodiments, the system comprises a transfer vector that encodes a therapeutic polynucleotide. The cells may be within a mammal, including a mammal that has a medical condition of any kind. The medical condition may or may not a genetic disease, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

For a more complete understanding of the present invention, reference is now made to the following descriptions taken in conjunction with the accompanying drawing, in which:

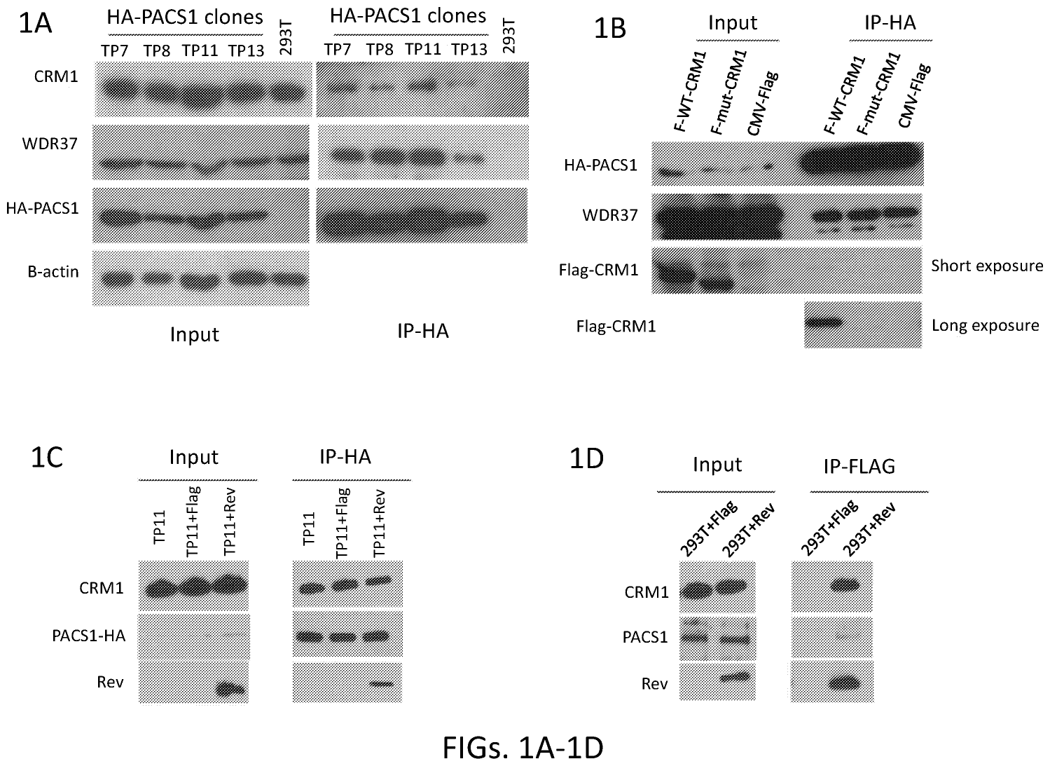

FIGS. 1A-1D-—CRM1 and Rev co-immunoprecipitate with PACS1. Four clonal 293T cell lines expressing an HA-PACS1 cDNA were generated as described in the text. 1A. Cell extracts were prepared from HA-PACS1 cell lines (TP7, TP8; TP11,TP13) and parental 293T cells, Immunoprecipitations of cell extracts were performed with an HA antibody; cell extracts (Input) and immunoprecipitates (IP-HA) were examined in an immunoblot for levels of the indicated proteins. 1B. The TP11 HA-PACS1 cell line was transfected with an expression plasmid for wild type Flag-CRAM1 (F-WT-CRM1), mutant Flag-CRM1 (F-mut-CRM1; deletion of residues 510-595), or parental Flag vector (CMV-Flag). Extracts were prepared and immunoprecipitations with HA antibody were performed; cell extracts (Input) and immunoprecipitates (IP-HA) were examined in an immunoblot. 1C. The TP11 HA-PACS1 cell line was transfected with a Flag-Rev expression plasmid (Rev) or parental Flag expression plasmid (Flag), cell extracts were prepared and immunoprecipitations were performed with the HA antibody; cell extracts (Input) and immunoprecipitates (IP-HA) were examined in an immunoblot, using the HA antibody to detect HA-PACS1 and Flag antibody to detect Flag-Rev. 1D. 293T cells were transfected with Flag-Rev expression plasmid (Rev) or parental Flag expression plasmid (Flag), cell extracts were prepared and immunoprecipitates were performed with a Flag antibody; cell extracts (Input) and immunoprecipitates were examined in immunoblots, using a PACS1 polyclonal antiserum to detect endogenous PACS1 and Flag antibody to detect Rev.

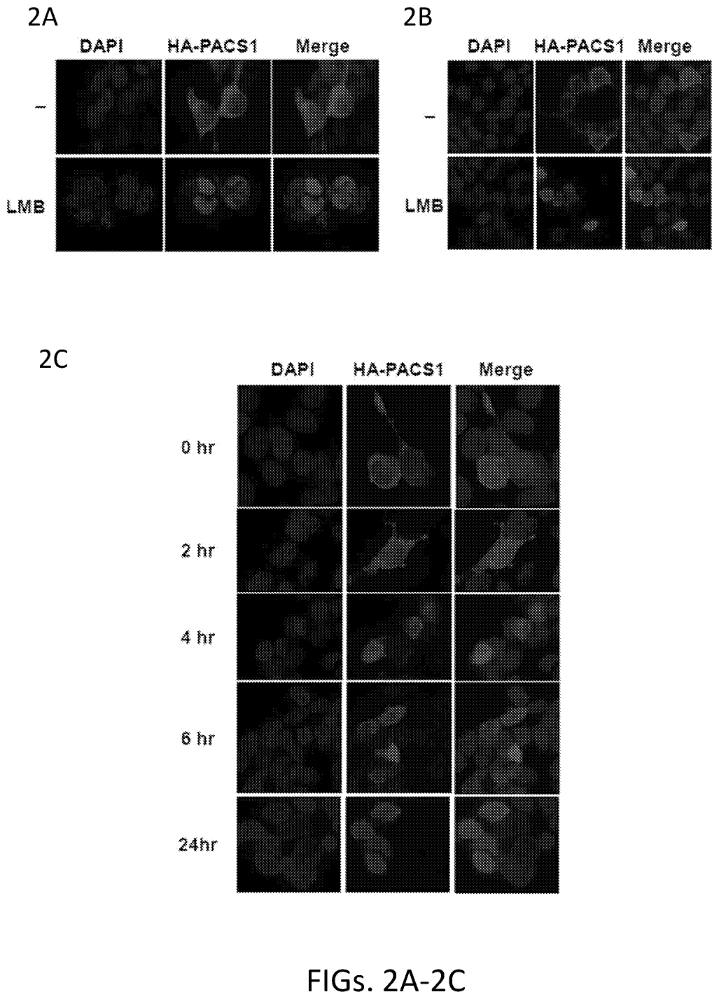

FIGS. 2A-2C—Leptomycin B treatment results in PACS1 localization from cytoplasm to nucleus. 2A. HA-PACS1 TP11 cells were incubated with or without Leptomycin B (LMB; 10 ng/ml) for 24 hours and examined by immunofluorescence using an HA-antibody. 2B. 293T cells were transfected with an HA-PACS1 expression plasmid, treated with or without 10 ng/ml LMB for 24 hours, and examined by immunofluorescence with an anti HA-antibody. 2C. 293T cells were transfected with an HA-PACS1 expression plasmid, treated with 10 ng/ml Leptomycin B for the indicated time, and examined by immunofluorescence with an anti HA-antibody.

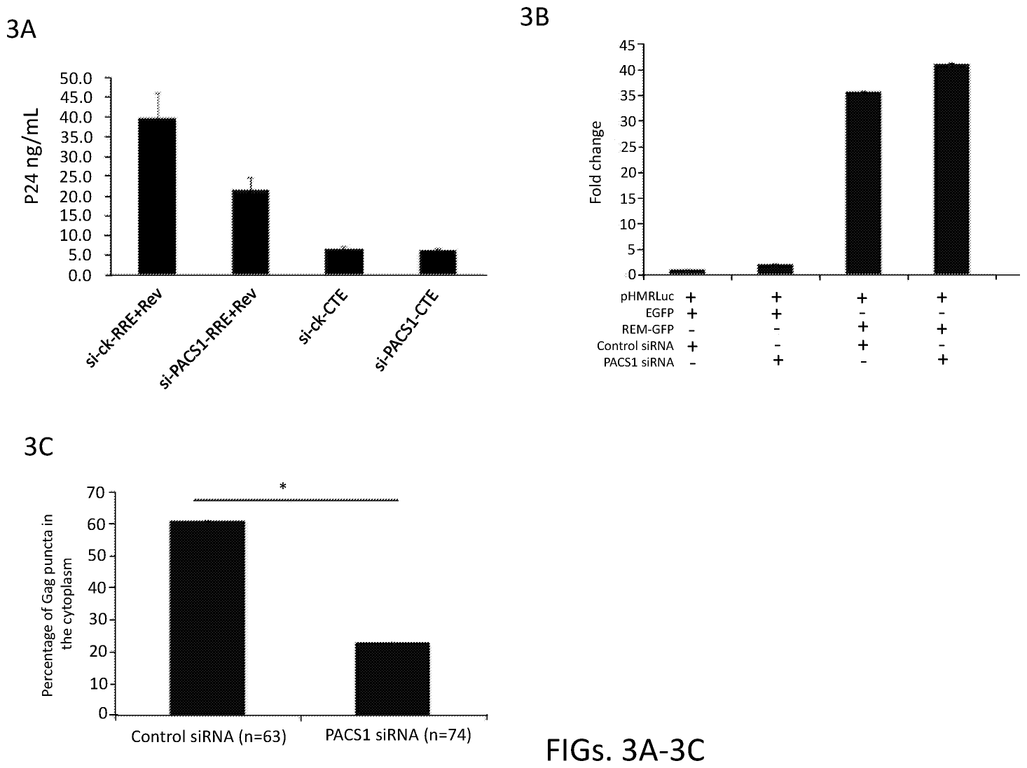

FIGS. 3A-3C—PACS1 siRNA depletions specifically inhibit Rev-RRE nuclear export pathway. 3A. 293T cells were transfected with control siRNAs or siRNAs against PACS1, followed by transfection 24 hours later with a pCMVGagPol-RRE+Rev-expression plasmid or a pCMVGagPol-CTE4X expression vector. Supernatants were collected 48 hours later and p24 levels were quantified by ELISAs. 3B. 293T cells were transfected with control, siRNAs or siRNAs against PACS1, followed by transfection 24 hours later as indicated with pHMRLuc (firefly Luciferase containing the Rem Response element), pCMV-Luc expressing Renilla Luciferase, and EGFP or RemGFP expression plasmids. Renilla and firefly Luciferase activities were measured in cell lysates at 24 hrs. post-transfection. Relative renilla Luciferase values were calculated by normalizing to firefly luciferase activity and setting the value to 1.0 in Control siRNA treated cells. 3C. 293T cultures were transfected with control siRNAs or siRNAs against PACS1 and 24 hours later transfected with pCMVGagPol-RRE and pCMVRev-Flag expression vectors. At 48 hrs. post-transfection, cells were fixed, stained with fluorescent Gag specific RNA probes and imaged by deconvolution microscopy as described previously (Budhiraja, et al., 2015). The percentage of cells containing Gag puncta in the cytoplasm were calculated from the total number of cells containing Gag puncta. *p<0.05 in a paired students t-test.

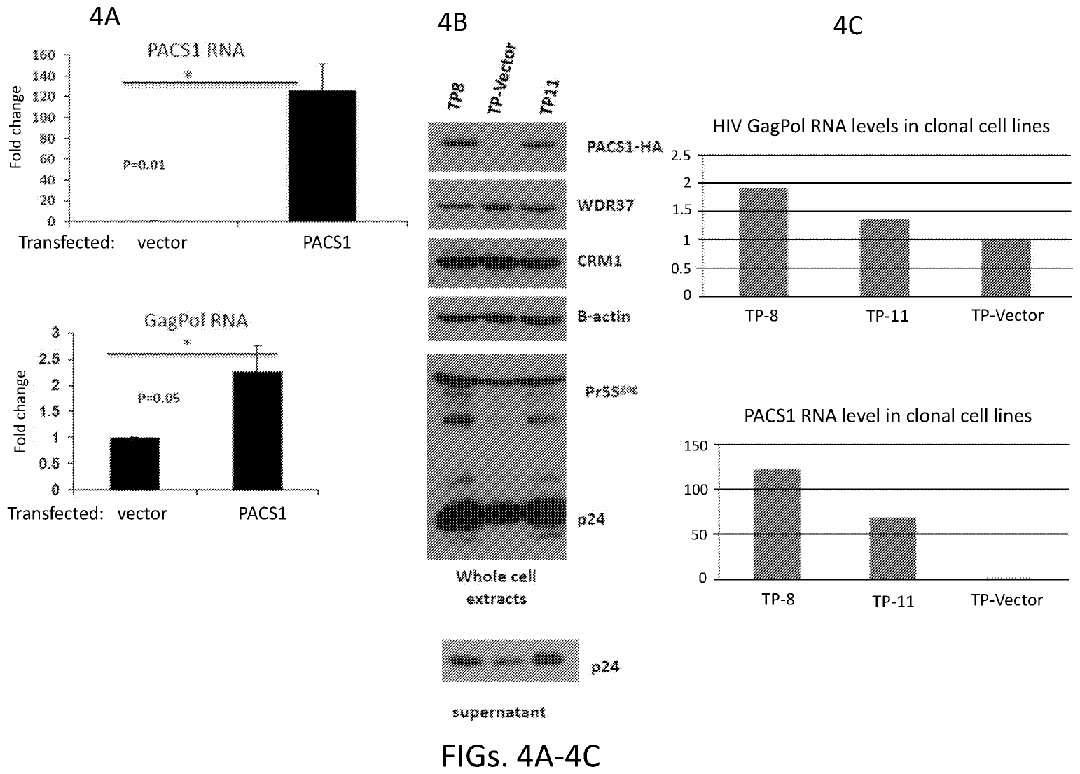

FIGS. 4A-4C—Over-expression of PACS1 enhances nuclear export of unspliced HIV-1 RNA. 4A. Cultures of 293T cells were transfected with a pNL4-3-GFP proviral plasmid and either an HA-PACS1 expression vector or parental vector. At 48 hours post-transfection, total cellular RNA was extracted. PACS1 RNA (top panel) and HIV GagPol RNA (bottom panel) levels were quantified by qRT-PCR. Levels of PACS1 and GagPol RNA in vector transfected cells were assigned an arbitrary value of 1.0. 4B. HA-PACS1 clonal cell lines TP8 and TP11 and control TP-Vector cell line were infected with an HIV-Luciferase reporter virus. Cell extracts were prepared at 48 hours post-infection and levels of indicated proteins were evaluated in an immunoblot. The level of PACS1 was evaluated with a polyclonal antiserum which detects both HA-PACS1 and endogenous PACS1. The supernatant from cells was collected at 48 hours post-infection and levels of p24 evaluated in an immunoblot. 4C. RNA was extracted (48 hours post-infection) from a portion of infected cultures described in panel B and GagPol RNA and PACS1 RNA levels were quantified by qRT-PCR. Levels of GagPol RNA and PACS1 RNA in TP-vector cells were assigned an arbitrary value of 1.0.

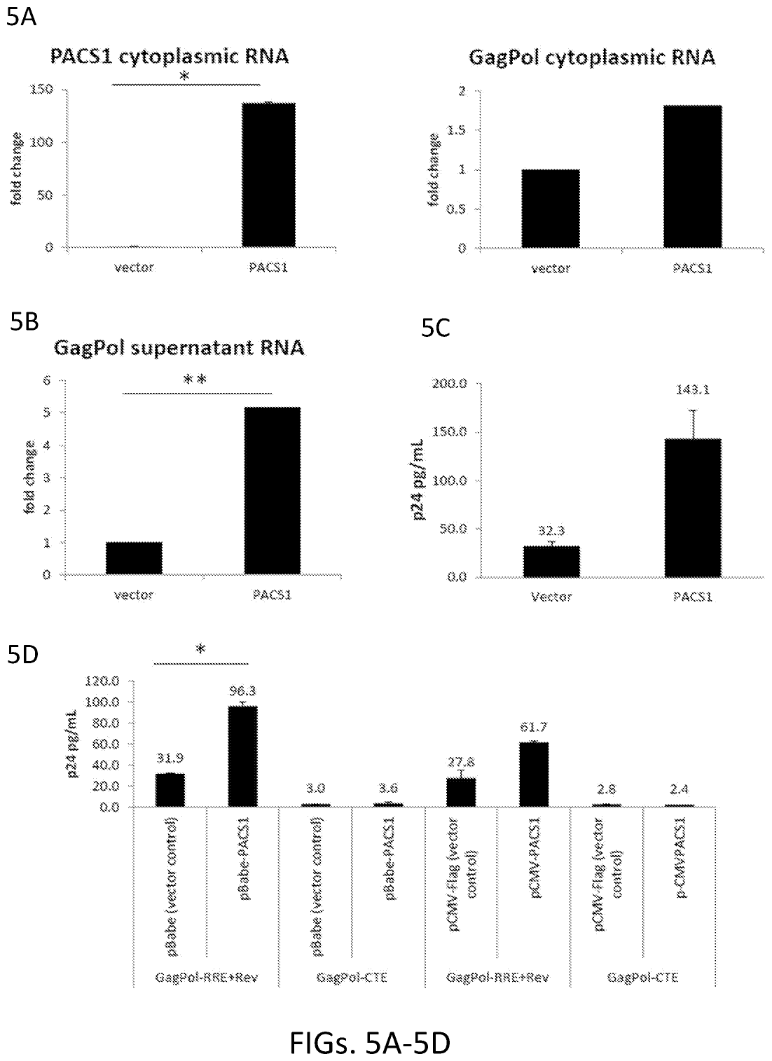

FIGS. 5A-5D—Over-expression of PACS1 stimulates HIV nuclear RNA export and p24 expression. 5A,5B. Cultures of 293T cells were transfected with either an HA-PACS1, expression vector or parental vector, At 24 hours post-transfection, cells were infected with HIV-1 NL4-3-Luciferase virus. At three days (72 hours) post-infection, cells and culture supernatants were collected. Total protein was extracted from a portion of the cells and cytoplasmic RNA was extracted from the remaining portion of cells; RNA was extracted from the supernatants. Cytoplasmic PACS1 RNA and HIV-1 GagPol RNA levels were quantified by real-time qRT-PCR, and normalized to GAPDH RNA. GagPol RNA in culture supernatants was quantified by real-time RT-PCR and normalized to the total protein of cultures. The levels of PACS1 and GagPol RNA in vector transfected cells were assigned an arbitrary value of 1.0; level of PACS1 RNA in PACS1-transfected cells is presented relative to this 1.0 value. 5C. Cultures of 293T cells were co-transfected with HIV-1 ⁻NL4-3-Luciferase proviral plasmid, a VSV-G expression plasmid, pRL-TK (Renilla luciferase reporter plasmid), and either an HA-PACS1 expression vector or parental vector. At 48 hours post-transfection, total p24 levels were quantified by ELISA. 5D. Cultures of 293T cells were co-transfected with either pCMV GalPol-RRE+Rev expression plasmid or pCMV-GagPol-CTE reporter plasmid. These reporter plasmids were co-transfected with a pBabe HA-PACS1 expression vector (retroviral), pBabe parental vector, pCMV vector, or pCMV-HA-PACS1 expression plasmid. At 48 hours post-transfection, p24 levels in culture supernatants were quantified by ELISAs. Values that were significantly different by a paired Student's t test are indicated by a bar and asterisks as follows:*, P<0.05; **, P<0.005.

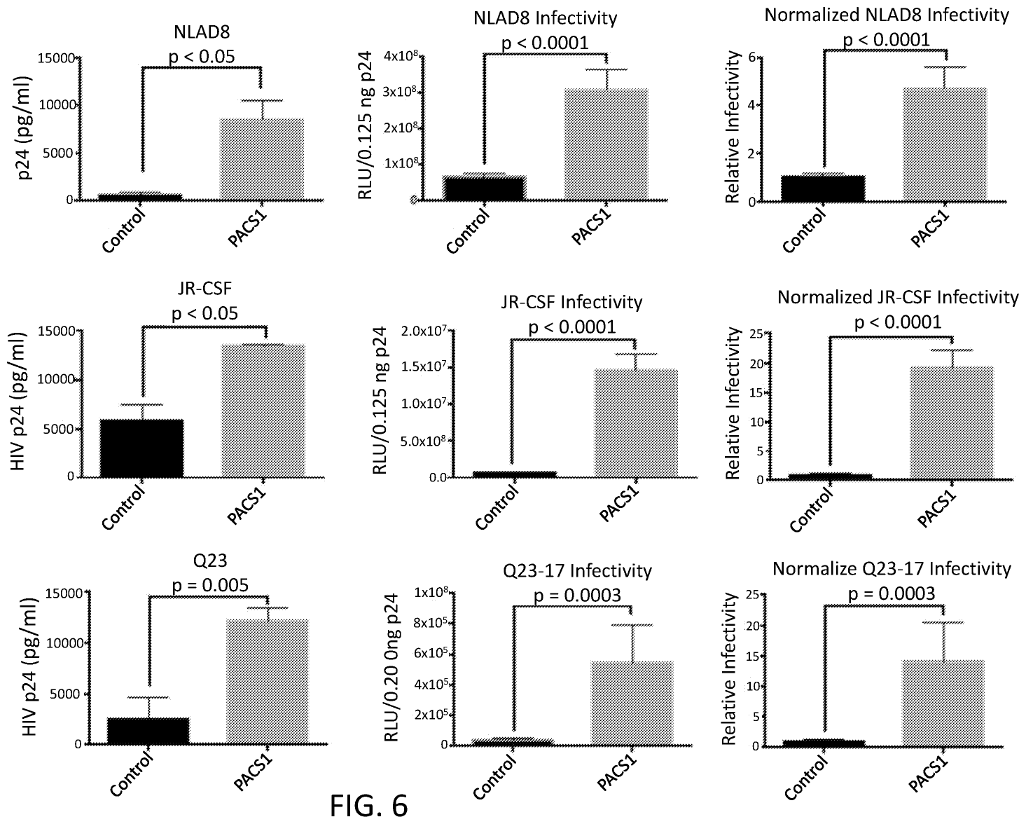

FIG. 6—Over-expression of PACS1 increases HIV-1 virion infectivity.

Cultures of 293T cells were co-transfected with PACS1 expression plasmid or parent vector plus proviral plasmids NLAD8, JR-CSF, or Q23-17. Three days later, culture supernatants were collected and p24 levels quantified by ELISAs (left panel). Transfections were done in triplicate and the average is shown with error bar. Equal amounts of p24 from culture supernatants were used to infect TZM-b1 cells and Luciferase expression was measured (middle panel). In right panel, Luciferase in Control was arbitrarily assigned a value of 1.0 and Luciferase in PACS1 samples shown relative to Control, Data was analyzed with Student's t test to demonstrate statistical significance.

### DETAILED DESCRIPTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

The term “vector” as used herein is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g, YACs). The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

### II. General Embodiments of the Disclosure

Embodiments of the disclosure encompass systems, methods, and compositions that improve upon lentiviral production and expression. In particular embodiments, the disclosure provides systems, methods, and compositions that enhance the amounts and/or infectivity of lentiviral vectors, including Rev-dependent lentiviral vectors, that leads to more robust utilization of the vectors.

The present disclosure demonstrates that PACS1 shuttles between the nucleus and cytoplasm and shows that it can be co-immunoprecipitated with CRM1 and Rev. SiRNA depletion experiments indicated that PACS1 is specific for the CRM-Rev-RRE nuclear export pathway and not the CRM1 export pathway of the Rem protein of Mouse Mammary Tumor Virus (MMTV), or the Constitutive Transport Element (CTE) export pathway of Mason-Pfizer Monkey Virus (MPMV). Over-expression of PACS1 stimulates nuclear export of unspliced HIV-1 RNA and accumulation of viral particles in the culture supernatants, indicating that PACS1 can be limiting for Rev HIV-1 replication. Unexpectedly, over-expression of PACS1 potently increases the infectivity of HIV-1 virions. Thus, the data indicate that in addition to its roles in Furin localization and down-regulation of MHC I by Nef, PACS1 has two additional distinct roles in HIV-1 replication—as a Rev co-factor and a co-factor involved in virion infectivity. This indicates that PACS1, and at least in some cases PACS2, are useful for production of Rev-dependent lentiviral vectors. The present disclosure encompasses systems of the vectors, methods for producing the vectors, using the vectors, and components of the vectors.

### III. Compositions of the Disclosure

Embodiments of the disclosure include the production of lentiviral vectors, components thereof, and the resultant lentiviral vectors themselves. In particular embodiments, PACS1 and/or PACS2 are present during production of the vectors and, as such, the production occurs in an enhanced manner over the production occurring in the absence of PACS1 and/or PACS2.

In particular embodiments, standard lentiviral processing methods may be utilized except for the addition of PACS1 and /or PACS2.

Lentiviruses have high mutation and recombination rates, so the likelihood that HIV could self-replicate and be produced during vector manufacturing by recombination is a serious safety concern. To reduce that probability, essential genes are separated into different plasmids, and the four viral accessory genes (vif, vpr, vpu and nef) may be deleted.

Several components are essential to generate a lentiviral vector, including: (1) a lentiviral backbone (which may be referred to herein as a transfer vector, transfer vector plasmid or lentiviral construct), wherein it includes long terminal repeats (LTRs) and the Packaging Signal Psi (Ψ); (2) a transgene of interest: e.g., a cDNA, miRNA, or shRNA cloned into the backbone (for example, a therapeutic polynucleotide); (3) at least one helper plasmid comprising packaging and envelope plasmid(s), and (4) a packaging cell line in which the viral vector production takes place. The transfer vector with the transgene and helper plasmids are transiently transfected into a packaging cell line (such as HEK-293 cells, for example) where the lentiviral vector gets assembled.

Several generations of HIV lentiviral vectors have been produced, and any of them may be utilized in the present disclosure. In specific embodiments, the vector system that is utilized is the highest generation of the vectors. In all three generations the envelope gene is usually heterologous, i.e., from a different virus, such as VSV-G (not an HIV gene).

A first-generation system includes a packaging system with all .HIY genes except for the env gene (usually heterologous) that is included in another vector. For example, the system may include a transfer vector that encodes the desired transgene, a packaging plasmid encoding gag, poi, tat, rev and accessory proteins, and an envelope plasmid encoding a heterologous env.

In second-generation systems, researchers discovered that the four HIV accessory genes—vif, vpr, vpu, and nef—were not required for HIV replication in immortalized cell lines. In second-generation systems, the four accessory genes were eliminated leaving the gag and pol reading frames and the tat and rev genes. In general, lentiviral vectors with a wild-type 5′ LTR need the 2nd generation packaging system because they need tat for activation.

Third-generation systems (also referred to as self-inactivating (SIN) systems), the 3′ LTR is modified, with tat being eliminated and rev provided in a separate plasmid. Because the HIV promoter in the 5′ LTR depends on tat, a vector without tat needs to have its wild-type promoter replaced with a heterologous enhancer/promoter to ensure transcription (for example, the promoter could be either viral (like CMV) or cellular (like EF1-α)).

During production of the lentiviral vectors, PACS1 and/or PACS2 are present either in the form of an expressible polynucleotide or in a protein form, and they may be present in whole or as a functional fragment thereof. When fragments of PACS1 and /or PACS2 are utilized, they are functional to enhance viral production compared to when the vectors are produced in their absence. Such quantitation of production may be measured by methods described elsewhere herein or by any means known in the art.

In specific embodiments, PACS1 and/or PACS2 are human, although in alternative cases they may be from another mammal, such as a rat or mouse, for example.

In specific embodiments, an example of a PACS1 polynucleotide is located at GenBank® Accession No. NM_018026 (SEQ ID NO:1). In specific embodiments, an example of a PACS1 polypeptide produced by polynucleotides of the present disclosure is located at GenBank® Accession No. NP_060496 (SEQ ID NO:2).

In some cases, a PACS1 and/or PACS2 polynucleotide encodes the entirety of the corresponding protein, although in certain cases the polynucleotide encodes part of the corresponding protein. The PACS1 and/or PACS2 polynucleotide may encode a fragment of the corresponding protein, including one lacking the N-terminus, the C-terminus, or both.

In some cases, the PACS1 polynucleotide is at least or no more than 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 nucleotides in length. In specific embodiments, the PACS1 polynucleotide is a functionally active derivative thereof and is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1.

In specific embodiments, an example of a PACS2 polynucleotide is located at GenBank® Accession No. AY320284 (SEQ ID NO:3). In specific embodiments, an example of a PACS2 polynucleotide produced by polynucleotides of the present disclosure is located at GenBank® Accession No. AAQ83882 (SEQ ID NO:4).

In some cases, the PACS1 polynucleotide encodes a polypeptide that is at least or no more than 950, 900, 850, 800, 750, 700, 650, 600, 550, 500, 450, 400, 350, 300, 250, 200, 150, 100, 75, 50, or 25 amino acids in length. In specific embodiments, the PACS1 polypeptide is a functionally active derivative thereof and is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ NO:2. A functionally active derivative of a PACS1 polypeptide may comprise 1, 2, 3, 4, 5, 6, 7, 8, 9, 10, or more substitutions, including at least conservative substitutions, for example.

In some cases, the PACS2 polynucleotide is at least or no more than 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 nucleotides in length. In specific embodiments, the PACS2 polynucleotide is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ II) NO:3.

In some cases, the PACS2 polynucleotide encodes a polypeptide that is at least or no more than 880, 850, 800, 750, 700, 650, 600, 550, 500, 450, 400, 350, 300, 250, 200, 150, 100, 75, 50, or 25 amino acids in length. In specific embodiments, the PACS2 polypeptide is a functionally active derivative thereof and is at least 70, 71, 72, 73, 74, 75, 76, 77, 78, 79, 80, 81, 82, 83, 84, 86, 87, 88, 89, 90, 91, 92, 93, 94, 95, 96, 97, 98, or 99% identical to SEQ ID NO:4. A functionally active derivative of a PACS2 polypeptide may comprise 1, 2, 3, 4, 5, 6, 7, 8, 9, 10, or more substitutions, including at least conservative substitutions, for example.

In specific embodiments, PACS1 and/or PACS2 are codon-optimized.

Any vectors referred to herein may comprise one or more regulatory regions (which may be a “promoter”) that are control sequence(s) that are a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a regulatory region is in a correct functional location and/or orientation in relation to a nucleic acid sequence to control transcriptional initiation and/or expression of that sequence. The regulatory region(s) employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

### IV. Methods of Production

Embodiments of the disclosure include methods of producing Rev-dependent lentiviral vectors in the presence of PACS1 and/or PACS2. Such conditions enhance HIV-1 RNA expression and viral production and enhance HIV-1 infectivity. Although this may occur by any mechanism, in specific embodiments the function of Rev is enhanced to allow for enhanced viral production and HIV-1 infectivity.

In specific embodiments, PACS1 and/or PACS2 are present in polynucleotide in a form that is expressible, although in alternative embodiments they are present in protein form. They may be present in the system in whole or in fragments. In some cases only PACS1 is utilized, where in other cases only PACS2 is utilized.

In some cases the PACS1 and/or PACS2 polynucleotide is comprised in a vector, and the vector may or may not be a vector that encodes one or more other components required for production of lentiviral vectors. The PACS1 and/or PACS2 polynucleotide may be present on the transfer vector, the packaging vector, and/or the envelop vector. The vector that comprises the PACS1 and/or PACS2 polynucleotide may be a plasmid or may be a viral vector.

Lentiviral vector production can include addition of an expression plasmid for PACS1 and/or PACS2 to transiently express the PACS1 and/or PACS2 protein in cell lines that produce the lentiviral vector. In other embodiments, cell lines can be generated that constitutively over-express PACS1 or PACS2; such cell lines can be used to produce the lentiviral vectors.

### V. Methods of Use

In particular embodiments, the lentiviral vectors produced by the methods of the disclosure are utilized for a research application or for a therapeutic application. A target cell for which the vector is transduced may or may not be a dividing cell.

In cases wherein the lentiviral vectors produced by the methods of the disclosure are utilized for research purposes, the vectors may harbor a polynucleotide of interest for which a function of an expressed product is desired to be known. The vector may be transfected into any type of cell for assaying a particular outcome, for example. The vector may be combined with other compositions for analysis of multiple components or interaction thereof, for example. Cell lines can be generated that over-express PACS1 and/or PACS2.

In cases wherein the lentiviral vectors produced by the methods of the disclosure are employed for therapeutic purposes, the vectors may harbor a polynucleotide of interest for which a function of an expressed product provides therapy to an individual in need thereof. The vector may provide therapy or prevention for one or more medical conditions for an individual in need thereof. The individual may be of any age or condition. The medical condition may be of any kind, including cancer, heart disease, stroke, diabetes, kidney disease, infection of any kind (including viral, bacterial, fungal, and so forth; influenza and pneumonia are examples), Alzheimer's Disease, bronchitis, emphysema, asthma, genetic diseases in which a therapeutic gene is beneficial, and so forth. The medical condition may or may not be a genetic disease, for example. The therapeutic polynucleotide utilized in the vector may or may not be of human origin. The target cell for the lentiviral vector may or may not be a diseased cell.

### VI. Kits of the Disclosure

Any of the compositions described herein may be comprised in a kit. In a non-limiting example, a vector that encodes part or all of PACS1 and/or PACS2 may be comprised in a kit. The kit may or may not include other components for lentiviral vector production, such as transfer vector(s) and/or component(s) thereof; packaging vector(s) and/or component(s) thereof; and/or envelope vector(s) and/or component(s) thereof. The kit also may include one or more reagents for recombinant technology, such as enzymes, buffers, nucleotides, primers, etc. Primers to amplify PACS1 and/or PACS2 may be provided in the kit.

In particular cases, a therapeutic polynucleotide of which the vector will harbor for delivery to an individual in need thereof may be present in the kit. In some cases, one or more primers to amplify a therapeutic polynucleotide may be provided in the kit. Primers to amplify one or more particular therapeutic polynucleotides may be provided in the kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition(s) and any other reagent containers in close confinement for commercial sale. Such containers may include injection or blow-molded plastic containers into which the desired vials are retained, for example.

When the components of the kit are provided in one and/or more liquid solutions, the liquid solution may be an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the vector(s) or component(s) thereof are placed, preferably, suitably allocated. The kits may also comprise a second container means for containing a sterile, pharmaceutically acceptable buffer and/or other diluent.

Irrespective of the number and/or type of containers, the kits of the disclosure may also comprise, and/or be packaged with, an instrument for assisting with the injection/administration and/or placement of the ultimate vector within the body of an animal. Such an instrument may be a syringe, pipette, forceps, and/or any such medically approved delivery vehicle, for example.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Plasmids, siRNAs, and antibodies. The pCMVGagPol-RRE and pCMV-RevFlag plasmids have been described previously and were a kind gift from Marie-Louise Hammarskjöld (University of Virginia). The pHMRLuc (Luciferase) and RemGFP (GFP stands for green fluorescent protein) plasmids were kindly provided by Japelin Dudley (University of Texas, Austin). PACS1 (sc-106348) and control (sc-37007) small interfering RNAs (siRNAs) were purchased from Santa Cruz Biotechnology, PACS1 siRNAs are a pooled mixture of three 19-25 nt siRNAs specific for the PACS1 gene. PACS1 antiserum (ab56072) was from Abcam; anti-HA antibody was from Santa Cruz Biotechnology (sc-7392); anti-CRM1 antibody (ST1100) and Flag antibody (F1804) were from Millipore and Sigma-Aldrich, respectively.

Transfections. For siRNA depletions, 293T cells were transfected with 30 pmol of siRNAs in 6-well culture dishes using Lipofectamine RNAimax (Life Technologies) according to the manufacturer's instructions. At 24 hr. after the siRNA transfections, cultures were transfected with 1.25 ug of pCMVGagPol-RRE plasmid and 1.25 ug of pCMV-RevFlag plasmid or 2.5 ug pCMV-MPMV (CTE) plasmid. Culture supernatants were harvested 24 hr after the plasmid transfection for p24 enzyme-linked immunosorbent assays (ELISAs) using the Zeptometrix ELISA kit, For some PACS1 over-expression experiments, 293T cells were transfected with an HA-PACS1 expression vector or parental vector. At 24 hours post-transfection, infected the transfected cells with NL4-3 HIV-1 virus. At 4 days (96 hours) post-infection, culture supernatant, and total cytoplasmic RNA were extracted. PACS1 RNA and HIV-1 GagPol RNA levels were quantified by qRT-PCR. For additional PACS1 over-expression experiments, 293T cells were co-transfected with 2 ug NL4-3-Luc proviral plasmid, lug VSV G expression plasdmid, 0.1 ug pRL-TK (wildtype Renilla luciferase (Rluc) control reporter vectors), and either an HA-PACS1 expression vector (pCMV) or parental vector (pCMV-Flag) in 6-well culture dishes using Lipofectamine®2000 (Life Technologies) according to the manufacturer's directions. At 48 hours post-transfection, total p24 levels were analyzed by Zeptometrix ELISA kit. In additional PACS1 over-expression experiments, 293T cells were co-transfected with 1.5 ug of pCMVGagPol-RRE plasmid and 1.5 ug of pCMV-RevFlag plasmid or 3 ug pCMV-MPMV (CTE) plasmid, and either an 1 ug HA-PACS1 expression vector (pBabe-PACS1-HA) or lug parental vector (pBabe) in 6-well culture dishes using Lipofectamine®2000 (Life Technologies) according to the manufacturer's directions. At 72 hours post-transfection, total p24 levels were analyzed by Zeptometrix ELISA kit.

Immunoprecipitations and immunoblots. Immunoprecipitations were performed using anti-Flag M2 affinity gel (A2220) from Sigma-Aldrich or Pierce-anti-HA agarose from Thermo Scientific using the manufacturer's protocol. Briefly, cultures transfected with Flag-tagged exression plasmids were lysed 48 h after transfection with EBC lysis buffer (Tris-HCl 50 mM, NaCl 120 mM, and 0.5% NP-40, pH=8.0); 20 ul of the anti-Flag M2 affinity gel or anti-HA agarose was washed with lysis buffer three times and incubated with the cell lysates overnight at 4° C. Following the incubation, beads were washed with lysis butler, and bound proteins were eluted by mixing and heating the beads in sample loading buffer for 5 min at 95° C. Samples were spun in a rnicrocentrifuge, and immunoprecipitates were loaded on a 7 to 12% Ms gradient gel (Bio-Rad). Gels were transferred to nitrocellulose membranes and blocked with 5% nonfat dry milk (NFDM) for an hour and probed with the appropriate antibodies.

Fluorescent in situ hybridization (FISH). Gag-specific Stellaris RNA fluorescent in situ hybridization (FISH) probes labeled with Quasar 670 fluorophore were obtained from Biosearch Technologies; the Gag probes are a pool of 40 individual probes. For FISH, the cells were fixed with fixation buffer (3.7% formaldehyde in phosphate-buffered saline [PBS]) for 20 min at room temperature followed by permeabilization with 70% ethanol for 48 h. The cells were washed once with wash buffer (10% formarnide in 2×SSC [1×SSC is 0.15 M NaCl plus 0.015 M sodium citrate]) and incubated with the probe in hybridization buffer (100 mg/ml dextran sulfate and 10% formatnide in 2×SSC) for 4 h at 37° C. Nonspecifically bound probes were removed by incubating the cells with wash buffer for 30 min at room temperature. Nuclei were stained with 4′, 6′-diamidino-2-phenvlindole (DAPI) and fixed for microscopy using Vectashield HardSet mounting medium (Vector Laboratories). Cells were analyzed using the DeltaVision (deconvolution) image restoration microscope in the Baylor College of Medicine Integrated Microscopy Core laboratory.

Immunoflorescence. For Leptomycin B experiments (LMB), 2931T cells were transfected with 400 ng of pCMV-PACS1-HA in 24-well culture dishes using Lipofectamine®2000 (Life Technologies) according to the manufacturer's instructions. At 24 hr. after plasmid transfections, the cultures were treated with 10 ng/mL LMB. After the indicated times of LMB treatment, cells were fixed with fixation buffer (4% paraformaldehyde in phosphate-buffered saline[PBS]) for 30 min at room temperature followed by permeabilization with 0.25 Triton-X-100 in PBS. The cells were blocked with 5% nonfat dry milk for 2 h, and then incubated with the primary antibody-anti-HA (Santa Cruz Biotechnology) overnight at 4° C. followed by Alexa Fluor® 594 secondary antibody (A-11037, Life Technologies). For DAPI stains, cells were treatemed with 5 ug/mL DAPI (D9542, Sigma-Aldrich) for 10 minutes before mounting. Images were taken using a deconvolution microscope at integrated Microscopy Core Laboratory in Baylor College of Medicine.

### Example 2

### PACS1 Co-Immunoprecipitates with Crm1 and Hiv-1 Rev

In order to study the role of PACS1 in RNA export, cell lines were generated that express PACS1 with an HA epitope tag. To generate these cell lines, 293T cells were transduced with an MLV-based retroviral vector encoding an HA-tagged PACS1 protein and a puromycin-resistance gene. Clonal cell lines were isolated by limiting dilution of puromycin-resistant cells; immunoblot analysis verified that four HA-PACS1 cell lines were established by this method (termed T7, T8, T11, and T13; FIG. 1A). Cell lysates were prepared from the cell lines and parental 2931′ cells and immunoprecipitations were performed with an anti-HA antibody. Immunoblot analysis of the immunoprecipitation products demonstrated that WDR37 (WD repeat domain 37) co-immunoprecipitated with HA-PACS1 as expected (FIG. 1A); although the functions of WDR37 are unknown, WDR37 was previously identified as a partner of PACS1 in the HeLa nuclear complexome (Malovannaya, et al., 2011), CRM1 also co-immunoprecipitated. with HA-PACS1 from extracts of each of the HA-PACS1 cell lines, also in agreement with the nuclear cotnplexome data which reported that PACS1 and CRM1 are found in a protein complex (Malovannaya, et al., 2011).

To examine the specificity of the association between PACS1 and CRM1, 293T cells were transfected with a wild type or mutant Flag-CRM1 expression plasmid. The mutant plasmid expresses a CRM1 protein with deletion of residues 510 to 595; this region of CRM1 forms a hydrophobic groove that is the binding site for NES-containing proteins (Dong, et al., 2009). As expected, WDR37 was present in immunoprecipitations with the HA-antibody (FIG. 1B). The wild type but not mutant CRM1 protein co-immunoprecipitated with HA-PACS1, demonstrating a requirement of the CRM1 hydrophobic groove for the association with PACS1.

It was next examined whether PACS1 could associate with the HIV-1 Rev protein. A Flag-tagged Rev expression plasmid was co-transfected into the Tp11 HA-PACS1 cell line, extracts were prepared and an immunoprecipitation was performed with an HA antibody (FIG. 1C). Both CRM1 and the Flag-Rev protein were observed in the HA-PACS1 immunoprecipitate. In an additional experiment, the Flag-Rev expression plasmid or parental Flag expression plasmid were transfected into 293T cells, cell extracts prepared, and immunoprecipitates were performed with a Flag antibody. An immunoblot analysis of the immunoprecipitates demonstrated that CRM1 and endogenous PACS1 co-immunoprecipitated with the Flag-Rev protein (FIG. 1D). These data indicate that PACS1 associates with CRM1 and Rev in cells.

### Example 3

### PACS1 Shuttles between the Nucleus and Cytoplasm

PACS1 has been observed to be a predominantly cytoplasmic protein (Dikeakos, et al., 2012). Given the positive role of PACS1 in Rev function (Budhiraja, et al., 2015) and its association with CRM1 and Rev demonstrated in FIG. 1, it was considered that PACS1 may shuttle between the nucleus and cytoplasm via a CRM1-dependent mechanism. Indeed, a PACS1-GFP fusion protein has been observed to accumulate in the nucleus when cells are treated with the specific inhibitor Leptornycin B (LMB) (Atkins, et al., 2014). LMB inhibits CRM1 through alkylation of Cys 528 in the NES-binding site (Kudo, et al., 1999). To verify that PACS1 is LMB-sensitive, the TP11 HA-PACS1 cell line was treated with LMB, An indirect immunofluorescence analysis demonstrated that HA-PACS1 was predominantly a cytoplasmic protein in non-LMB-treated cells as expected (FIG. 2A). In cells treated with LMB, however, HA-PACS1 predominantly localized in the nucleus. To confirm this observation, 293T cells were transfected with an HA-PACS1 expression plasmid and PACS1 localization was examined with and without LMB treatment (FIG. 2B). In agreement with the observation in the TP11 cell line, HA-PACS1 was predominantly cytoplasmic in non-LMB-treated 293T cells, while it became predominantly nuclear in LMB-treated cells. A time course was also examined of HA-PACS1 localization after LMB treatment in 293T cells transfected with a HA-PACS1 expression plasmid (FIG. 1C). Prior to LMB treatment (0 hour), PACS1 was predominantly cytoplasmic. PACS1 began to accumulate in the nucleus at 4 hours post-treatment and was seen predominantly in the nucleus after 6 hours of LMB treatment. In specific embodiments, although PACS1 predominantly accumulates in the cytoplasm, it shuttles between the nucleus and cytoplasm in a CRM1-dependent mechanism.

### Example 4

### PACS1 Sirna Depletion Inhibits Rev-RRe But Not CTE RNA Nuclear Export or Mmtv Crm1-REM Nuclear Export

SiRNA depletions of PACS1 inhibited Rev nuclear export of unspliced HIV-1 RNA as assayed with a pCMV-GagPol-RRE reporter plasmid (Budhiraja, et al., 2015). To examine the specificity of PACS1 in RNA export, PACS1 siRNA depletions were used to evaluate the role of PACS1 in the CRM1-Rev export pathway, the Constitutive Transport Element (CTE) export pathway (Bray, et al., 1994), and the MMTV CRM1-Rem export pathway (Mertz, et al., 2005). Depletion of PACS1 inhibited CRM1-Rev nuclear export as assayed by p24 expression from a transfected pCMV-GagPol-RRE reporter plasmid and a Rev expression plasmid (FIG. 3A). A previous studied verified that these siRNAs were effective in reducing the level of PACS1 protein (Budhiraja, et al., 2015). In contrast, PACS1 siRNAs had no effect on the CTE pathway as assayed by p24 expression of a pCMV-GagPol-CTE reporter plasmid. Similar to the CTE pathway, PACS1 depletion had no effect of MMTV Rev nuclear export as assayed with the pHRMLuc MMTV reporter plasmid (FIG. 3B). To verify that PACS1 depletions inhibited nuclear export of unspliced HIV-1 transcripts, we performed fluorescent in situ hybridization (FISH) assays. Fluorescently labeled Gag RNA probes were used to determine the cellular localization of Gag transcripts expressed from the pCMV-GagPol-RRE reporter, as described recently (Budhiraja, et al., 2015). PACS1 depletion resulted in a significant reduction in the accumulation of Gag transcripts in the cytoplasm, in agreement with reduction of p24 expression from the reporter plasmid (FIG. 3C). Thus, PACS1 is a positive factor involved in HIV-1 Rev export of unspliced viral transcripts. Furthermore, PACS1 appears not to be involved in CTE-mediated nuclear export or MMTV CRM1-Rem nuclear export.5

### Example 5

### PACS1 Over-Expression Increase HIV-1 RNA Nuclear Export and P24 Expression

It was next considered if over-expression of PACS1 could stimulate nuclear export of unspliced HIV-1 transcripts. 293T cells were co-transfected with a pNL4-3-GFP proviral plasmid with either the HA-PACS1 expression vector or the parental vector. Total cellular RNA was extracted at 48 hours post-transfection and levels of PACS1 RNA and HIV-1 GagPol RNA were quantified by qRT-PCR (FIG. 4A). As expected, PACS1 RNA levels were greatly elevated in the HA-PACS1 transfected cells relative to cells transfected with the parent vector. Co-transfection of the HA-PACS1 expression plasmid increased unspliced GagPol RNA levels approximately 2.3-fold relative to the parental vector. These data indicate that over-expression of PACS1 can stimulate Rev nuclear export of unspliced viral transcripts.

The HA-PACS1 293T cell lines TP8 and TP11 (see FIG. 1) were utilized to examine the effect over-expressed PACS1 on expression of unspliced HIV-1 during viral infection. TP8, TP11, and control TP-Vector cells were infected with a VSV-pseudotyped HIV-1-Luciferase reporter virus. At 48 hours post-infection, cell extracts were prepared from a portion of the infected cells for immunoblot analysis, and RNA was extracted from another portion of infected cells; supernatants were also collected to examine the levels of p24 in culture supernatants. A polyclonal antiserum against PACS1 was used to examine the levels of both endogenous and HA-PACS1 in the different cell lines (FIG. 4B). PACS1 was significantly over-expressed in both the TP8 and TP11 cell lines; a longer exposure of the immunoblot in FIG. 4B was able to detect endogenous PACS1 in the TP-Vector cell extract . There was an increase in level of p24 in both cell extracts and culture supernatants from the TP8 and TP11 infected cells relative to the control TP-Vector infected cells. In agreement with the protein expression measurements, both PACS1 RNA and HIV-1 GagPol RNA were elevated in the TP8 and TP11 cell lines relative to the TP-Vector control (FIG. 4C).

The data presented in FIG. 4 indicate that HIV-1 RNA nuclear export and p24 expression are enhanced in cell lines that over-express PACS1. To confirm that over-expression of PACS1 enhances viral RNA export and p24 expression, experiments were performed in which PACS1 was over-expressed transiently from expression plasmids. Cultures of 293T cells were transfected with an HA-PACS1 expression plasmid or parental vector. At 24 hours-post transfection, cultures with infected with a VSV G pseudotyped HIV-1 NL4-3 luciferase virus. At three days post infection, culture supernatants were collected and cytoplasmic extracts were prepared. RNA was extracted from both supernatants and cytoplasmic extracts and Gag-Pol and PACS1 RNAs were quantified by real-time RT PCR. Transfection of the PACS1 expression plasmid resulted in a large increase in PACS1 cytoplasmic RNA as expected (FIG. 5A). Transfection of the PACS1 plasmid resulted in a 1.8-fold increase in cytoplasmic GagPol RNA (FIG. 5A) and a 5-fold increase in GagPol RNA in the culture supernatant (FIG. 5B).

In an additional experiment, cultures of 293T cells were co-transfected with the HA-PACS1 expression plasmid or parental vector plus an HIV-1 NL4-3 Luciferase proviral plasmid. At 48 hours-post transfection, levels of p24 in culture supernatants were quantified by ELISA (FIG. 5C). Over-expression of PACS1 resulted in a 4.4-fold increase in p24 levels, in agreement with the PACS1 enhancement of GagPol RNA levels in culture supernatants in the experiment shown in FIG. 5B.

PACS1 siRNA depletion does not inhibit CTE-directed RNA nuclear export (FIG. 3a). It was desired to determine if over-expression of PACS1 affects the CTE export pathway. Cultures of 293T cells were co-transfected with the CMV-GagPol-CTE reporter plasmid plus a retroviral-based (pBabe) PACS1 expression vector, a CMV-based PACS1 expression vector, or the parental vectors. The CMV-GagPol-RRE reported plus a Rev expression vector was included as a positive control. At 48 hours post-transfection, the level of p24 in culture supernatants was quantified by ELISA (FIG. 5D). Unlike PACS1 enhancement of p24 levels and GagPol RNA expression via the HIV-1 Rev-RRE pathway, over-expression of PACS1 by either expression vector had little effect on the CTE pathway. Over-expression of PACS1 from the pBabe vector enhanced p24 expression from the GagPol-RRE reporter (plus Rev) 3-fold and approximately 2-fold from the CMV-based vector. Over-expression of PACS1 specifically stimulates the HIV-1 Rev-RNA nuclear export pathway.

### Example 6

### PACS1 Over-Expression Increases HIV-1 Virion Infectivity

The data presented in FIGS. 4 and 5 indicate that over-expression of PACS1 can increase the level of GagPol RNA and p24 in culture supernatants expressed from derivatives of the NL4-3 HIV-1. To evaluate if over-expression of PACS1 can increase the level of virus produced from other HIV-1 strains, the PACS1 vector was transfected with three different proviral plasmids: NLAD8 (subtype A), macrophage-tropic derivative of provirus NL4-3 (Freed, et al., 1994); JR-CSF (subtype B), isolated from the CSF (Koyanagi, et al., 1987); Q23-17 (subtype A), isolated from an infected infant (Provine, et al., 2012). As shown in FIG. 6 (left panel), PACS1 over-expression increased virus production >10-fold for NLAD8, ˜3-fold for JR-CSF, and ˜6-fold for Q23-17. These increases agree well with the increase in viral RNA in the supernatant of infected cells that over-express PACS1 (FIG. 5B). To quantify viral infectivity, equal amounts of p24 from culture supernatants were used to infect TZM-b1 cells and Luciferase levels were measured (FIG. 6, middle panels). After normalization (setting Control levels as 1.0, right panels), PACS1 over-expression was found to increase virion infectivity ˜5-fold for NLAD8, ˜18-fold for JR-CSF, and ˜13-fold for Q23-17. These data indicate that as well as increasing viral production, PACS1 potently enhances virion infectivity. Thus, PACS1 affects multiple aspects of the HIV-1 replication cycle.

### Example 7

### Significance of Certain Embodiments

In this disclosure, PACS1 is shown to be involved in the nuclear export of incompletely spliced viral RNAs by the viral Rev protein. In addition, the inventors made the unexpected observation that over-expression of PACS1 potently increases virion infectivity. Previous studies reported that PACS1 associates with the viral Nef protein and is involved in down-regulation of MECI (Piquet, et al., 2000; Blagoveshchenskaya, et al., 2002; Dikeakos, et al., 2012). PACS1 may also have a role, albeit indirect, in Furin cleavage of the HIV-1 Envelope gp160 protein into gp41 and gp120, as PACS1 mediates localization of Furin to the trans-Golgi network (Wan, et al., 1998; Hallenberger, et al., 1992). Thus, PACS1 is a remarkable multi-tasking co-factor that participates in four distinct processes of the HIV-1 replication cycle-nuclear export of viral RNA, enhancement of virion infectivity, down-regulation of MHCI, and cleavage of the viral Envelope protein.

In the present disclosure, extensive data is provided indicating that PACS1 is a Rev co-factor. PACS1 can be co-immunoprecipitated with Rev and CRM1, and its over-expression stimulates the level of unspliced transcripts in the cytoplasm and in virions that bud into the culture supernatant. The disclosure confirms a previous report that PACS1 shuttles between the nucleus and cytoplasm, a property consistent with that of a Rev co-factor (Atkins, et al., 2014). PACS1 is also shown as specific for the Rev-CRM1 nuclear export pathway, as siRNA depletion of PACS1 has no effect on nuclear export via the MMTV Rem-CRM1 pathway or the MPMV Constitutive Transport Element pathway that utilizes the NXF1/NXT1 export pathway. It is perhaps not surprising that PACS1 does not have a role in the CTE-NXF1/NXT1 pathway, as live cell imaging has demonstrated that the Rev-CRM1 and CTE-NXF1 export pathways have distinct trafficking properties. Viral RNA exported via the Rev-CRM1 pathway traffics to the cytoplasm in a non-localized fashion, while RNA exported via the CTE-NXF1/NXT1 pathway traffics to microtubules in the cytoplasm (Pocock, et al., 2016). The role of PACS1 in the HIV-1 Rev-CRM1 export but not MMTV Rem-CRM1 export indicates that these two viral proteins utilize divergent CRM1-dependent pathways.

The inventors made the unexpected observation that over-expression of PACS1 can enhance virion infectivity of three different HIV-1 isolates: NLAD8 (subtype A), macrophage-tropic derivative of provirus NL4-3 (Koyanagi, et al., 1994); JR-CSF (subtype B), isolated from the CSF (Koyanagi, et al., 1987); Q23-17 (subtype A), isolated from an infected infant (Provine, et al., 2012). The mechanisms involved in enhanced infectivity are unknown at this time. SERINC3 and SERINC5 are recently identified restriction factors that our countered by the HIV-1 Nef protein (Rosa, et al., 2015; Usami, et al., 2015). However, initial experiments suggest that PACS1 over-expression does not down-regulate SERINC3 or SERINC5. Additionally, PACS1 over-expression does not affect Nef relocalization of SERINC5 from the plasma membrane to cytoplasmic clusters (Rosa, et al., 2015; Usami, et al., 2015). Initial experiments also suggest that over-expression of PACS1 does not affect the level of Envelope incorporated into HIV-1 virions, nor does it affect cleavage of gp160. In some embodiments, over-expression of PACS1 could result in the inactivation of an unidentified restriction factor that is incorporated into virions and decreases infectivity.

While lower metazoans encode a single PACS gene, higher metazoans encode two related PACS genes—PACS1 and PACS2. PACS1 and PACS2 are broadly expressed in all tissues examined. PACS1 is selectively enriched in peripheral blood lymphocytes and this may be of significance to HIV1 infection (Youker, et al, 2009). In higher eukaryotes, both PACS1 and PACS2 regulate membrane trafficking, including TGN localization (Youker, et al., 2009), and both proteins shuttle between the nucleus and cytoplasm ((Atkins, et al., 2014), FIG. 2). As shown here, PACS1. is involved in Rev-mediated nuclear RNA export, while PACS2 has been shown to regulate SIRT1-medidated deacetylation of p53 in the nucleus (Atkins, et al, 2014). Thus, both PACS1 and PACS2 are multi-functional proteins that are involved in both nuclear and cytoplasmic processes.

The finding that over-expression of PACS1 increases the level of Rev-dependent RNAs and virion infectivity has practical applications for production of lentiviral vectors. The studies indicate that the amounts and infectivity of Rev-dependent lentiviral vectors produced from transfected plasmids can be increased by the over-expression of PACS1.

SEQUENCES

SEQ ID NO: 1, human

| | |
|---|---|
| 1 | gcagctcgct ggctgctcgc gctcgggcag gcgggctgag gaggctgccg cgcccccgcc |
| 61 | gccgccgccg cgggggaagc ctgggagcca gatcggcgtc gcctcggcct ccgtaacccc |
| 121 | cgcctagccg ggccatggcg gaacgcggag gggcgggcgg tggtcccgga ggcgccgggg |
| 181 | gcggcagcgg ccagcgggga tccggggtcg cccagtcccc tcagcagccg ccgccgcagc |
| 241 | agcagcagca gcagccgccg cagcagccga cgccccccaa gctggcccag gccacctcgt |
| 301 | cgtcctcgtc cacctcggcg gcggctgcct cctcctcgtc ctcgtctacc tccacctcca |
| 361 | tggccgtggc ggtggcctcg ggctccgcgc ctcccggtgg cccggggcca ggccgcaccc |
| 421 | ccgccccggt gcagatgaac ctgtacgcca cctgggaggt ggaccggagc tcgtccagct |
| 481 | gcgtgcctag gctattcagc ttgaccctga agaaactcgt catgctaaaa gaaatggaca |
| 541 | aagatcttaa ctcagtggtc atcgctgtga agctgcaggg ttcaaaaaga attcttcgct |
| 601 | ccaacgagat cgtccttcca gctagtggac tggtggaaac agagctccaa ttaaccttct |
| 661 | cccttcagta ccctcatttc cttaagcgag atgccaacaa gctgcagatc atgctgcaaa |
| 721 | ggagaaaacg ttacaagaat cggaccatct tgggctataa gaccttggcc gtgggactca |
| 781 | tcaacatggc agaggtgatg cagcatccta atgaaggcgc actggtgctt ggcctacaca |
| 841 | gcaacgtgaa ggatgtctct gtgcctgtgg cagaaataaa gatctactcc ctgtccagcc |
| 901 | aacccattga ccatgaagga atcaaatcca agctttctga tcgttctcct gatattgaca |
| 961 | attattctga ggaagaggaa gagagtttct catcagaaca ggaaggcagt gatgatccat |
| 1021 | tgcatgggca ggacttgttc tacgaagacg aagatctccg gaaagtgaag aagacccgga |
| 1081 | ggaaactaac ctcaacctct gccatcacaa ggcaacctaa catcaaacag aagtttgtgg |
| 1141 | cactcctgaa gcggtttaaa gtttcagatg aggtgggctt tgggctggag catgtgtccc |
| 1201 | gcgagcagat ccgggaagtg gaagaggact tggatgaatt gtatgacagt ctggagatgt |
| 1261 | acaaccccag cgacagtggc cctgagatgg aggagacaga aagcatcctc agcacgccaa |
| 1321 | agcccaagct caagcctttc tttgagggga tgtcgcagtc cagctcccag acggagattg |
| 1381 | gcagcctcaa cagcaaaggc agcctcggaa aagacaccac cagccctatg gaattggctg |
| 1441 | ctctagaaaa aattaaatct acttggatta aaaaccaaga tgacagcttg actgaaacag |
| 1501 | acactctgga aatcactgac caggacatgt ttggagatgc cagcacgagt ctggttgtgc |
| 1561 | cggagaaagt caaaactccc atgaagtcca gtaaaacgga tctccagggc tctgcctccc |
| 1621 | ccagcaaagt ggagggggtg cacacacccc ggcagaagag gagcacgccc ctgaaggagc |
| 1681 | ggcagctctc caagccccta agtgagagga ccaacagttc cgacagcgag cgctccccag |
| 1741 | atctgggcca cagcacgcag attccaagaa aggtggtgta tgaccagctc aatcagatcc |
| 1801 | tggtgtcaga tgcagccctc ccagaaaatg tcattctggt gaacaccact gactggcagg |
| 1861 | gccagtatgt ggctgagctg ctccaggacc agcggaagcc tgtggtgtgc acctgctcca |
| 1921 | ccgtggaggt ccaggccgtg ctgtccgccc tgctcacccg gatccagcgc tactgcaact |
| 1981 | gcaactcttc catgccgagg ccagtgaagg tggctgctgt gggaggccag agctacctga |
| 2041 | gctccatcct caggttcttt gtcaagtccc tggccaacaa gacctccgac tggcttggct |
| 2101 | acatgcgctt cctcatcatc cccctcggtt ctcaccctgt ggccaaatac ttggggtcag |
| 2161 | tcgacagtaa atacagtagt tccttcctgg attctggttg gagagatctg ttcagtcgct |
| 2221 | cggagccacc agtgtcagag caactggacg tggcagggcg ggtgatgcag tacgtcaacg |
| 2281 | gggcagccac gacacaccag cttcccgtgg ccgaagccat gctgacttgc cggcataagt |
| 2341 | tccctgatga agactcctat cagaagttta ttcccttcat tggcgtggtg aaggtgggtc |
| 2401 | tggttgaaga ctctccctcc acagcaggcg atggggacga ttctcctgtg gtcagcctta |
| 2461 | ctgtgccctc cacatcacca ccctccagct cgggcctgag ccgagacgcc acggccaccc |
| 2521 | ctccctcctc cccatctatg agcagcgccc tggccatcgt ggggagccct aatagcccat |
| 2581 | atggggacgt gattggcctc caggtggact actggctggg ccaccccggg gagcggagga |
| 2641 | gggaaggcga caagagggac gccagctcga agaacaccct caagagtgtc ttccgctcag |
| 2701 | tgcaggtgtc ccgcctgccc catagtgggg aggcccagct ttctggcacc atggccatga |
| 2761 | ctgtggtcac caaagaaaag aacaagaaag ttcccaccat cttcctgagc aagaaacccc |
| 2821 | gagaaaagga ggtggattct aagagccagg tcattgaagg catcagccgc ctcatctgct |
| 2881 | cagccaagca gcagcagact atgctgagag tgtccatcga tggggtcgag tggagtgaca |
| 2941 | tcaagttctt ccagctggca gcccagtggc ccacccatgt caagcacttt ccagtgggac |
| 3001 | tcttcagtgg cagcaaggcc acctgaggcc ctgtctccca gccactttcc ctcctggcac |
| 3061 | tgccaccagc ctcaccgcct gcgggcaggg ggaggccagc aggcccgggc ccagcacccc |
| 3121 | ttccctggca ccagggtctg cctctcactc gcccaggtcc cgaaggacac tgccacaggg |
| 3181 | acgccttccc tcccctcccc tccagcccac ccctgcacag cccctcctcc ttcccgcttt |
| 3241 | tccccttctc cctcctgctc caggcccaag gcgtgttggt tttgccttct ggtgcccata |
| 3301 | gtcccctgga ctgagtcccc caggccttcc ttcacccgac ttccaaactc ttccttgtgg |
| 3361 | tatcagtttc cttctcggaa atgagaaagc tggaatcctg gtccccagca ggagagccta |
| 3421 | gtcctccccc agcccctcca gccaccaggg tgtcctctag gatgcagctg ccagatccac |
| 3481 | tcactctgct gcctccagca ggacccaagg ccactttcaa ctcttatggg gactccacc |
| 3541 | tgccccagag cttctcaagg gagggtaagg gggcaccctg agcccacagg acccctactt |
| 3601 | cacagctcac aggggcagga ggcagctccc ctgcctccag gaccctgttg ctatggtgac |
| 3661 | acagcgtttc taggacagag gggcctccca gtctcccccc accacccgtg cacgacttcc |
| 3721 | tcaccacccc caggttccct gcagatgtcg tgtgtgtcct gagtgtttct ttggttcttt |
| 3781 | gcacgccaag tctcttggtt gtaccatgtg acacaccctg tgcactggtc gctgtcttcg |
| 3841 | tggcttccac ccttgttaat gatgctcctg cctctgcctc ccagcccctc acccagcaca |
| 3901 | gctctgcctg gacttggaga gatgggaggc agacccccac caccatacat gctgtctgtg |
| 3961 | gcccctcaga cattctgttt catctcccat tcatctccct cctcccaccg tgtcagtttt |
| 4021 | tctgcctttc cctgctctgt tcttccccct ccttaggccc cagcctgggc ccagacccat |
| 4081 | cctcccagcc aggtttccct ccagcaggct ccttccctcc ctgtcacctc cctctcacca |
| 4141 | acccggggtc tgagcccctc attcctgacc gtccgtgttc tcaggagtgg ttgaggacac |
| 4201 | agggccccag cccagccctc tgcacccccc agcccggcca tctgcgcccc acagcccctt |
| 4261 | tggagctttt ctcttgtcct ctcactcctt cccagaagtt tttgcacaga acttcatttt |
| 4321 | gaaagtgttt ttctcattct ccatacctcc cccaagctct cctccagccc ttcccagggc |
| 4381 | tcagccctgc tgtcctgagc gtctcctggg ccagagagag gagatggggg tgggagggac |
| 4441 | tgagttgatg ttgggttttt cattcaataa attggtgatt tcttaccgac tgcaaaaaaa |
| 4501 | aaaaaaaaaa a |

SEQ ID NO: 2, human
MAERGGAGGGPGGAGGGSGQRGSGVAQSPQQPPPQQQQQQPPQQ
PTPPKLAQATSSSSSTSAAAASSSSSSTSTSMAVAVASGSAPPGGPGPGRTPAPVQMN
LYATWEVDRSSSSCVPRLFSLTLKKLVMLKEMDKDLNSVVIAVKLQGSKRILRSNEIV
LPASGLVETELQLTFSLQYPHFLKRDANKLQIMLQRRKRYKNRTILGYKTLAVGLINM
AEVMQHPNEGALVLGLHSNVKDVSVPVAEIKIYSLSSQPIDHEGIKSKLSDRSPDIDN
YSEEEEESFSSEQEGSDDPLHGQDLFYEDEDLRKVKKTRRKLTSTSAITRQPNIKQKF
VALLKRFKVSDEVGFGLEHVSREQIREVEEDLDELYDSLEMYNPSDSGPEMEETESIL
STPKPKLKPFFEGMSQSSSQTEIGSLNSKGSLGKDTTSPMELAALEKIKSTWIKNQDD
SLTETDTLEITDQDMFGDASTSLVVPEKVKTPMKSSKTDLQGSASPSKVEGVHTPRQK
RSTPLKERQLSKPLSERTNSSDSERSPDLGHSTQIPRKVVYDQLNQILVSDAALPENV
ILVNTTDWQGQYVAELLQDQRKPVVCTCSTVEVQAVLSALLTRIQRYCNCNSSMPRPV
KVAAVGGQSYLSSILRFFVKSLANKTSDWLGYMRFLIIPLGSHPVAKYLGSVDSKYSS
SFLDSGWRDLFSRSEPPVSEQLDVAGRVMQYVNGAATTHQLPVAEAMLTCRHKFPDED
SYQKFIPFIGVVKVGLVEDSPSTAGDGDDSPVVSLTVPSTSPPSSSGLSRDATATPPS
SPSMSSALAIVGSPNSPYGDVIGLQVDYWLGHPGERRREGDKRDASSKNTLKSVFRSV
QVSRLPHSGEAQLSGTMAMTVVTKEKNKKVPTIFLSKKPREKEVDSKSQVIEGISRLI
CSAKQQQTMLRVSIDGVEWSDIKFFQLAAQWPTHVKHFPVGLFSGSKAT
SEQ ID NO: 3, human

| | |
|---|---|
| 1 | ggtggacccc cacgactctc ccggcccttg cccgcggctc ctcgcgcacc ggcgggcggg |
| 121 | gacgccccgt gaggcgccgt cggaggaagc gcgcgcgcac ctcacttccg ggcgggcggg |
| 181 | cgccggcggc gatttggacc cgaggcggcg agctggcgcc ccgcccagcc aatcggcggc |
| 241 | gcggcgcggg tcggagggcg ccgggcgcgc gcggggcggc cgggggcgcg cggggcgcgt |
| 301 | gcggggcgcc gggcggggcg gggcggacgg ccgcagctcg tcgccgcccg cgggcctgtc |
| 361 | cgacgccggg gcccggcccg tcccctccgc cgcccggcag ccatgtgacc gcgccgccgc |
| 421 | cctccgcgcg cccggcccgc ccgccgcgcg tccgcggccc ggccgcagcc ccaggccgcc |
| 481 | gagggagcgg cggggccggc gccatggccg agcgaggccg cctcggcctc cccggcgcgc |
| 541 | tcggcgcgct caacacgccc gtgcccatga acctgttcgc cacctgggag gtggacggct |
| 601 | ccagccccag ctgcgtgccc aggttgtgca gcctgactct gaagaagctg gtggtcttca |
| 661 | aggagctgga gaaggagctg atctccgtgg tgatcgctgt caagatgcag ggctccaaac |
| 721 | gaatcctgcg gtcccatgag attgtgctgc cccccagtgg acaagtggag acagacctgg |
| 781 | ccctgacctt ctccttgcag tatcctcact tcttgaagag ggaaggcaac aagcttcaga |
| 841 | tcatgctgca gcgcagaaag cgctacaaga acagaaccat cctgggctac aagacgctgg |
| 901 | ccgcgggctc catcagcatg gctgaggtga tgcaacaccc gtctgaaggt ggccaggtgc |
| 961 | tgagcctctg cagcagcatc aaggaggccc ccgtcaaggc ggccgagatc tggatcgcct |
| 1021 | ccctgtccag ccagcccatt gaccacgaag acagcaccat gcaggccggc cccaaggcca |
| 1081 | agtccacgga taactactcc gaggaggagt atgagagctt ctcctccgag caggaggcca |
| 1141 | gtgacgacgc cgtgcagggg caggacttgg acgaggacga ctttgacgtg gggaagccga |
| 1201 | agaagcagcg gagatcgatt gtaagaacga cgtccatgac caggcaacag aacttcaagc |
| 1261 | agaaagtggt agcgctgctg cggaggttca aagtgtccga cgaggtcctg gactcggagc |
| 1321 | aggaccctgc ggagcacatc cccgaggcag aggaggacct ggacctcctg tatgacaccc |
| 1381 | tggacatgga gcaccccagc gacagcggcc ccgacatgga ggatgacgac agcgtcctca |
| 1441 | gcacccccaa gccgaagctg cggccatact ttgaaggcct gtcgcactcg agctcgcaga |
| 1501 | cggagattgg gagcatccac agcgcccgca gccacaagga gcccccaagc ccggctgacg |
| 1561 | tgcccgagaa gacgcggtcc ctgggaggca ggcagccgag cgacagtgtc tctgacacgg |
| 1621 | tggccctcgg tgtgccaggc ccgagggagc accctggaca gcctgaggac agccccgagg |
| 1681 | ctgaggcctc caccctggat gtgttcacgg agaggctgcc gcccagcggg aggatcacca |
| 1741 | agacagagtc ccttgtcatc ccctccacca ggtccgaagg gaagcaggct ggccgacggg |
| 1801 | gccggagcac atccttgaag gagcggcagg cagcacggcc ccagaatgag cgggccaaca |
| 1861 | gcctggacaa cgagcgctgc ccggacgccc ggagccagct acagatcccc aggaagactg |
| 1921 | tgtatgacca gctcaaccac atcctcatct ccgatgacca gcttcccgaa aacatcatcc |
| 1981 | ttgtcaacac ctccgactgg caggggcagt tcctctccga cgtcctgcag aggcacacgc |
| 2041 | tccccgtggt gtgcacgtgc tctcctgcgg acgtccaggc ggccttcagc accatcgtct |
| 2101 | cacggataca gagatactgc aactgcaatt cccagccccc gacccccgtg aagatcgccg |
| 2161 | tggcgggagc gcagcattac ctcagtgcca tcctgcggct ctttgtggag cagctgtccc |
| 2221 | acaagacacc cgactggctc ggctacatgc gcttcctggt catcccactg ggctcccacc |
| 2281 | ccgtggccag gtacctaggc tccgtggact accgctacaa caacttcttc caggacctgg |
| 2341 | cctggagaga cctgttcaac aagctggagg cccagagtgc ggtacaggac acgccagaca |
| 2401 | ttgtgtcacg catcacgcag tacatcgcag gggccaactg tgcccaccag ctccccatcg |
| 2461 | cagaggccat gctgacctac aagcagaaga gccctgacga agagtcctcc caaaagttca |
| 2521 | ttccctttgt cggggttgtg aaggttggaa ttgtggagcc atcttcggcc acatcaggcg |
| 2581 | actcggacga cgcggcoacc tcgggctctg gcacgctctc atccaccccg ccgtccgcat |
| 2641 | ctcctgcggc caaggaggcc tcacccaccc cgccctcctc cccgtcggtg agcggaggcc |
| 2701 | tgtcctcccc cagccagggt gtcggcgccg agctgatggg gctgcaggtg gactactgga |
| 2761 | cggcagcaca gcctgcggac aggaagaggg acgccgagaa gaaggacctg cctgtcacca |
| 2821 | aaaacacgct caagtgcact ttccggtccc tccaggtcag caggctgccc agcagcggcg |
| 2881 | aggctgcagc cacgcccacc atgtccatga ccgtggtcac caaggagaag aacaagaagg |
| 2941 | tgatgtttct gcccaagaaa gcgaaggaca aggacgtgga gtctaagagc cagtgcattg |
| 3001 | agggcatcag ccggctcatc tgcactgcca ggcagcagca gaacatgctg cgggtcctca |
| 3061 | tcgacggcgt ggagtgcagc gacgtcaagt tcttccagct ggccgcgcag tggtcctcgc |
| 3121 | acgtgaagca cttccccatc tgcatcttcg gacactccaa ggccaccttc tag |

SEQ ID NO: 4, human
MAERGRLGLPGALGALNTPVPMNLFATWEVDGSSPSCVPRLCSL
TLKKLVVFKELEKELISVVIAVKMQGSKRILRSHEIVLPPSGQVETDLALTFSLQYPH
FLKREGNKLQIMLQRRKRYKNRTILGYKTLAAGSISMAEVMQHPSEGGQVLSLCSSIK
EAPVKAAEIWIASLSSQPIDHEDSTMQAGPKAKSTDNYSEEEYESFSSEQEASDDAVQ
GQDLDEDDFDVGKPKKQRRSIVRTTSMTRQQNFKQKVVALLRRFKVSDEVLDSEQDPA
EHIPEAEEDLDLLYDTLDMEHPSDSGPDMEDDDSVLSTPKPKLRPYFEGLSHSSSQTE
IGSIHSARSHKEPPSPADVPEKTRSLGGRQPSDSVSDTVALGVPGPREHPGQPEDSPE
AEASTLDVFTERLPPSGRITKTESLVIPSTRSEGKQAGRRGRSTSLKERQAARPQNER
ANSLDNERCPDARSQLQIPRKTVYDQLNHILISDDQLPENILLVNTSDWQGQFLSDVL
QRHTLPVVCTCSPADVQAAFSTIVSRIQRYCNCNSQPPTPVKIAVAGAHHYLSAILRL
FVEQLSHKTPDWLGYMRFLVIPLGSHPVARYLGSVDYRYNNFFQDLAWRDLFNKLEAQ
SAVQDTPDIVSRITQYIAGANCAHQLPIAEAMLTYKQKSPDEESSQKFIPFVGVVKVG
IVEPSSATSGDSDDAAPSGSGTLSSTPPSASPAAKEASPTPPSSPSVSGGLSSPSQGV
GAELMGLQVDYWTAAQPADRKRDAEKKDLPVTKNTLKCTFRSLQVSRLPSSGEAAATP
TMSMTVVTKEKNKKVMFLPKKAKDKDVESKSQCIEGISRLICTARQQQNMLRVLIDGV
ECSDVKFFQLAAQWSSHVKHFPICIFGHSKATF

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

All patents and publications cited herein are hereby incorporated by reference in their entirety herein.
Arrigo S J, Chen I S. 1991. Rev is necessary for translation but not cytoplasmic accumulation of HIV-1 vif, vpr, and env/vpu 2 RNAs. Genes Dev, 5:808-819,
Atkins K M, Thomas L L, Barroso-Gonzalez J, Thomas L, Auclair S, Yin J, Kang H, Chung J H, Dikeakos J D, Thomas G. 2014. The multifunctional sorting protein PACS-2 regulates SIRT1-mediated deacetylation of p53 to modulate p21-dependent cell-cycle arrest. Cell Rep. 8:1545-1557. doi: S2211-1247(14)00633-0 [pii]; 10.1016/j.celrep.2014.07.049 [doi].
Blagoveshchenskaya A D, Thomas L, Feliciangeli S F, Hung C H, Thomas G. 2002. HIV-1 Nef downregulates MHC-I by a PACS-1- and PI3K-regulated ARF 6 endocytic pathway. Cell 111:853-866. doi: S0092867402011625 [pii].
Blissenbach M, Grewe B, Hoffmann B, Brandt S, Uberla K. 2010. Nuclear RNA export and packaging functions of HIV-1 Rev revisited. J. Virol. 84:6598-6604. doi: JVI.02264-09 [pii]; 10.111.28/JVI.02264-09 [doi].
Brandt S, Blissenbach M, Grewe B, Konietzny R, Grunwald T, Uberla K. 2007. Rev proteins of human and simian immunodeficiency virus enhance RNA encapsidation. PLoS Pathog.3:e54. doi: 06-PLPA-RA-0444R3 [pii]; 10.1371/journal.ppat.0030054 [doi].
Bray M, Prasad S, Dubay J W, Hunter E, Jeang K T, Rekosh D, Hammarskjold ML. 1994. A small element from the Mason-Pfizer monkey virus genome makes human immunodeficiency virus type 1 expression and replication Rev-independent. Proc Natl Acad Sci U S A. 91:1256-1260.
Budhiraja S, Liu H, Couturier J, Malovannaya A, Qin J, Lewis DE, Rice AP. 2015. Mining the human complexome database identifies RBM14 as an XPO1-associated protein involved in HIV-1 Rev function. J Virol 89:3557-3567. doi :JVI.03232-14 [pii]; 10.1128/JVI.03232-14 [doi].
Bushman F, Lewinski M, Ciuffi A, Barr S, Leipzig J, Hannenhalli S, Hoffmann C. 2005. Genome-wide analysis of retroviral DNA integration. Nat Rev. Microbiol. 3:848-858.
D' Agostino D M, Felber B K, Harrison J E, Pavlakis G N. 1992. The Rev protein of human immunodeficiency virus type 1 promotes polysomal association and translation of gag/pol and vpu/env mRNAs. Mol. Cell Biol. 12:1375-1386.
Dikeakos J D, Thomas L, Kwon G, Elferich J, Shinde U, Thomas G. 2012. An interdomain binding site on HIV-1 Nef interacts with PACS-1 and PACS-2 on endosomes to down-regulate MHC-I. Mol Biol Cell 23:2184-2197.
Dong X, Biswas A, Seel K E, Jackson L K, Martinez R, Gu H, Chook Y M. 2009. Structural basis for leucine-rich nuclear export signal recognition by CRM1. Nature 458:1136-1141, doi: nature07975 [pii];10.1038/nature07975 [doi].
Fang J, Kubota S, Yang B, Zhou N, Zhang H, Godbout R, Pomerantz R J. 2004. A DEAD box protein facilitates HIV-1 replication as a cellular co-factor of Rev. Virology. 330:471-480.
Fernandes J D, Booth D S, Frankel A D. 2016. A structurally plastic ribonucleoprotein complex mediates post-transcriptional gene regulation in HIV-1. Wiley. Interdiscip. Rev. RNA. doi: 10.1002/wrna. 1342 [doi].
Freed E O, Martin M A. 1994. HIV-1 infection of non-dividing cells. Nature 369:107-108. doi: 10.1038/369107b0 [doi].
Hailenberger S, Bosch V, Angliker H, Shaw E, Klenk H D, Garten W. 1992. Inhibition of furin-mediated cleavage activation of HIV-1 glycoprotein gp160. Nature 360:358-361. doi: 10,1038/360358a0 [doi],
Kimura T, Hashimoto I, Nishikawa M, Fujisawa J I. 1996. A role for Rev in the association of HIV-1 gag mRNA with cytoskeletal beta-actin and viral protein expression. Biochimie 78:1075-1080. doi: S0300-9084(97)86732-6 [pii].
Koyanagi Y, Miles S, Mitsuyasu R T, Merrill J E, Vinters H V, Chen I S. 1987. Dual infection of the central nervous system by AIDS viruses with distinct cellular tropisms. Science 236:819-822.
Kudo N, Matsumori N, Taoka H, Fujiwara D, Schreiner E P, Wolff B, Yoshida M, Horinouchi S. 1999. Leptomycin B inactivates CRM1/exportin 1 by covalent modification at a cysteine residue in the central conserved region. Proc. Natl. Acad. Sci. U. S. A 96:9112-9117.
Kula A, Guerra J, Knezevich A, Kleva D, Myers M P, Marcello A. 2011. Characterization of the HIV-1 RNA associated proteome identifies Matrin 3 as a nuclear cofactor of Rev function. Retrovirology. 8:60. doi: 1742-4690-8-60 [pii]; 10.1186/1742-4690-8-60 [doi].
Li J, Tang H, Mullen T M, Westberg C, Reddy T R, Rose D W, Wong-Staal F. 1999. A role for RNA helicase A in post-transcriptional regulation of HIV type 1. Proc. Natl. Acad. Sci. U. S. A 96:709-714.
Malovannaya A, Lanz R B, Jung S Y, Bulynko Y, Le N T, Chan D W, Ding C, Shi Y, Yucer N, Krenciute G, Kim B J, Li C, Chen R, Li W, Wang Y, O'Malley B W, Qin J. 2011. Analysis of the human endogenous coregulator complexome. Cell. 145:787-799.
Mertz J A, Simper M S, Lozano M M, Payne S M, Dudley J P. 2005. Mouse mammary tumor virus encodes a self-regulatory RNA export protein and is a complex retrovirus. J. Virol. 79:14737-14747. doi: 79/23/14737 [pii]; 10.1128/JVI.79.23.14737-14747.2005 [doi].
Piguet V, Wan L, Borel C, Mangasarian A, Demaurex N, Thomas G, Trono D. 2000. HIV-1 Nef protein binds to the cellular protein PACS-1 to downregulate class I major histocompatibility complexes. Nat. Cell Biol 2:163-167. doi: 10.1038/35004038 [doi].
Pocock G M, Becker J T, Swanson C M, Ahlquist P, Sherer N M. 2016. HIV-1. and M-PMV RNA Nuclear Export Elements Program Viral Genomes for Distinct Cytoplasmic Trafficking Behaviors. PLoS Pathog. 12:e1005565. doi: 10.1371/journal.ppat.1005565 [doi]; PPATHOGENS-D-15-02993 [pii].
Provine N M, Cortez V, Chohan V, Overbaugh J. 2012. The neutralization sensitivity of viruses representing human immunodeficiency virus type 1 variants of diverse subtypes from early in infection is dependent on producer cell, as well as characteristics of the specific antibody and envelope variant. Virology 427:25-33. doi :S0042-6822(12)00094-3 [pii]; 10.1016/j.viro1.2012.02.001 [doi].
Rosa A, Chande A, Ziglio S, De S, V, Bertorelli R, Goh S L, McCauley S M, Nowosielska A, Antonarakis S E, Luban J, Santoni F A, Pizzato M. 2015. HIV-1 Nef promotes infection by excluding SERINCS from virion incorporation. Nature 526:212-217. doi: nature15399 [pii]; 10.1.038/nature15399 [doi].
Shida H. 2012. Role of Nucleocytoplasmic RNA Transport during the Life Cycle of Retroviruses. Front Microbiol. 3:179. doi: 10.3389/fmicb.2012.00179 [doi].
Sloan K E, Gleizes P E, Bohnsack M T. 2016, Nucleocytoplasmic Transport of RNAs and RNA-Protein Complexes. J Mol. Biol 428:2040-2059. doi: S0022-2836(15)00546-X [pii]; 10.1016j.jmb,2015,09.023 [doi].
Usami Y. Wu Y, Gottlinger H G. 2015. SERINC3 and SERINCS restrict HIV-1 infectivity and are counteracted by Nef. Nature 526:218-223. doi: nature15400 [pii]; 10.1038/nature15400 [doi].
Wan L, Molloy S S, Thomas L, Liu G, Xiang Y, Rybak S L, Thomas G. 1998. PACS-1 defines a novel gene family of cytosolic sorting proteins required for trans-Golgi network localization. Cell 94:205-216. doi: S0092-8674(00)81420-8 [pii].
Yedavalli V S, Jeang K T. 2010, Trimethylguanosine capping selectively promotes expression of Rev-dependent HIV-1 RNAs. Proc. Natl. Acad. Sci. U. S. A 107:14787-14792. doi: 1009490107 [pii]; 10.1073/pnas.1009490107 [doi].
Yedavalli V S, Jeang K T. 2011. Matrin 3 is a co-factor for HIV-1 Rev in regulating post-transcriptional viral gene expression. Retrovirology. 8:61. doi: 1742-4690-8-61 [pii]; 10.1186/1742-4690-8-61 [doi].
Yedavalli V S, Neuveut C, Chi Kleiman L, Jeang K T. 2004. Requirement of DDX3 DEAD box RNA helicase for HIV-1 Rev-RRE export function. Cell 119:381-392.
Youker R T, Shinde U, Day R, Thomas G. 2009. At the crossroads of homoeostasis and disease: roles of the PACS proteins in membrane traffic and apoptosis. Biochem. J 421:1-15. doi: BJ20081016 [pii]; 10.1042/BJ20081016 [doi].
Zhang Q, Chen C Y, Yedavalli V S, Jeang K T. 2013. NEAT1 long noncoding RNA and paraspeckle bodies modulate HIV-1 posttranscriptional expression. MBio z1:e00596-12. doi: mBio.00596-12 [pii]; 10.11281mBio.00596-12 [doi].

1. A system for producing a lentiviral expression vector, comprising a vector that encodes Phosphofurin Acid Cluster Sorting protein 1 (PACS1) and/or Phosphofurin Acid Cluster Sorting protein 2 (PACS2).

2. The system of claim 1, wherein the lentiviral expression vector is Rev-dependent.

3. The system of claim 1, wherein the vector that encodes PACS1 and/or PACS2 encodes one or more lentiviral expression vector components.

4. The system of claim 1, wherein the vector that encodes PACS1 and/or PACS2 does not encode one or more lentiviral expression vector components.

5. The system of claim 1, wherein the vector that encodes PACS1 and/or PACS2 is a transfer vector for production of the Rev-dependent lentiviral expression vector.

6. The system of claim 1, wherein the vector that encodes PACS1 and/or PACS2 is not a transfer vector for production of the Rev-dependent lentiviral expression vector.

7. The system of claim 1, wherein the vector that encodes PACS1 and/or PACS2 is a packaging vector for production of the Rev-dependent lentiviral expression vector.

8. The system of claim 1, wherein the vector that encodes PACS1 and/or PACS2 is not a packaging vector for production of the Rev-dependent lentiviral expression vector.

9. The system of claim 1, wherein the vector that encodes PACS1 and/or PACS2 is an envelope vector for production of the Rev-dependent lentiviral expression vector.

10. The system of claim 1, wherein the vector that encodes PACS1 and/or PACS2 is not an envelope vector for production of the Rev-dependent lentiviral expression vector.

11. The system of claim 1, wherein the vector that encodes PACS1 and/or PACS2 is a plasmid.

12. The system of claim 1, wherein one or more regulatory regions for expression of the PACS1 and/or PACS2 are inducible.

13. The system of claim 1, wherein one or more regulatory regions for expression of the PACS1 and/or PACS2 are constitutive.

14. The system of claim 1, wherein one or more regulatory regions for expression of the PACS1 and/or PACS2 are tissue-specific.

15. The system of claim 1, wherein the system is comprised in or is configured to function in a cell.

16. The system of claim 15, wherein the cell is a eukaryotic cell.

17. The system of claim 1, wherein PACS1 comprises SEQ ID NO:2 or a functionally active derivative of fragment thereof.

18. The system of claim 17, wherein PACS1 is encoded by a polynucleotide comprising SEQ ID NO:1 or a functionally active derivative of fragment thereof.

19. The system of claim 1, wherein PACS2 comprises SEQ ID NO:4 or a functionally active derivative of fragment thereof.

20. The system of claim 19, wherein PACS2 is encoded by a polynucleotide comprising SEQ ID NO:3 or a functionally active derivative of fragment thereof.

21. The system of claim 1, wherein the system is housed in a cell that overexpresses PACS1 and/or PACS2.

22. A method of producing a Rev-dependent lentiviral expression vector, comprising the step of exposing a system that produces a Rev-dependent lentiviral expression vector to a vector that encodes Phosphofurin Acid Cluster Sorting protein 1 (PACS1) and/or Phosphofurin Acid Cluster Sorting protein 2 (PACS2), or two vectors each that encode one of PACS1 and PACS2, wherein the system that produces a Rev-dependent lentiviral expression vector comprises at least a transfer vector, one or more packing vectors, and an envelope vector, wherein the method occurs in a eukaryotic cell.

23. The method of claim 22, wherein an envelope protein encoded by the envelope vector is not an HIV envelope protein.

24. The method of claim 23, wherein the envelope protein is a Vesicular stomatitis virus (VSV-G) envelope protein or Zika virus envelope protein.

25. A method of transducing a target cell, comprising the step of exposing the target cell to a Rev-dependent lentiviral expression vector, wherein the Rev-dependent lentiviral expression vector was produced from a system that comprised a vector that encoded PACS1 and/or PACS2.

26. The method of claim 25, wherein the target cell is a human cell.

27. The method of claim 26, wherein the human cell is a diseased cell.

28. The method of claim 1, wherein the system comprises a transfer vector that encodes a therapeutic polynucleotide.

29. The method of claim 1, wherein the cells is within a mammal.

30. The method of claim 29, wherein the mammal has a medical condition.


SEQUENCE LISTING (4 sequences)

SEQ ID NO: 1 - DNA, 4511 bp, Homo sapiens
gcagctcgctggctgctcgcgctcgggcaggcgggctgaggaggctgccgcgcccccgccgccgccgccgcgggggaagcctgggagccagatcggcgtcgcctcggcctccgtaacccccgcctagccgggccatggcggaacgcggaggggcgggcggtggtcccggaggcgccgggggcggcagcggccagcggggatccggggtcgcccagtcccctcagcagccgccgccgcagcagcagcagcagcagccgccgcagcagccgacgccccccaagctggcccaggccacctcgtcgtcctcgtccacctcggcggcggctgcctcctcctcgtcctcgtctacctccacctccatggccgtggcggtggcctcgggctccgcgcctcccggtggcccggggccaggccgcacccccgccccggtgcagatgaacctgtacgccacctgggaggtggaccggagctcgtccagctgcgtgcctaggctattcagcttgaccctgaagaaactcgtcatgctaaaagaaatggacaaagatcttaactcagtggtcatcgctgtgaagctgcagggttcaaaaagaattcttcgctccaacgagatcgtccttccagctagtggactggtggaaacagagctccaattaaccttctcccttcagtaccctcatttccttaagcgagatgccaacaagctgcagatcatgctgcaaaggagaaaacgttacaagaatcggaccatcttgggctataagaccttggccgtgggactcatcaacatggcagaggtgatgcagcatcctaatgaaggcgcactggtgcttggcctacacagcaacgtgaaggatgtctctgtgcctgtggcagaaataaagatctactccctgtccagccaacccattgaccatgaaggaatcaaatccaagctttctgatcgttctcctgatattgacaattattctgaggaagaggaagagagtttctcatcagaacaggaaggcagtgatgatccattgcatgggcaggacttgttctacgaagacgaagatctccggaaagtgaagaagacccggaggaaactaacctcaacctctgccatcacaaggcaacctaacatcaaacagaagtttgtggccctcctgaagcggtttaaagtttcagatgaggtgggctttgggctggagcatgtgtcccgcgagcagatccgggaagtggaagaggacttggatgaattgtatgacagtctggagatgtacaaccccagcgacagtggccctgagatggaggagacagaaagcatcctcagcacgccaaagcccaagctcaagcctttctttgaggggatgtcgcagtccagctcccagacggagattggcagcctcaacagcaaaggcagcctcggaaaagacaccaccagccctatggaattggctgctctagaaaaaattaaatctacttggattaaaaaccaagatgacagcttgactgaaacagacactctggaaatcactgaccaggacatgtttggagatgccagcacgagtctggttgtgccggagaaagtcaaaactcccatgaagtccagtaaaacggatctccagggctctgcctcccccagcaaagtggagggggtgcacacaccccggcagaagaggagcacgcccctgaaggagcggcagctctccaagcccctaagtgagaggaccaacagttccgacagcgagcgctccccagatctgggccacagcacgcagattccaagaaaggtggtgtatgaccagctcaatcagatcctggtgtcagatgcagccctcccagaaaatgtcattctggtgaacaccactgactggcagggccagtatgtggctgagctgctccaggaccagcggaagcctgtggtgtgcacctgctccaccgtggaggtccaggccgtgctgtccgccctgctcacccggatccagcgctactgcaactgcaactcttccatgccgaggccagtgaaggtggctgctgtgggaggccagagctacctgagctccatcctcaggttctttgtcaagtccctggccaacaagacctccgactggcttggctacatgcgcttcctcatcatccccctcggttctcaccctgtggccaaatacttggggtcagtcgacagtaaatacagtagttccttcctggattctggttggagagatctgttcagtcgctcggagccaccagtgtcagagcaactggacgtggcagggcgggtgatgcagtacgtcaacggggcagccacgacacaccagcttcccgtggccgaagccatgctgacttgccggcataagttccctgatgaagactcctatcagaagtttattcccttcattggcgtggtgaaggtgggtctggttgaagactctccctccacagcaggcgatggggacgattctcctgtggtcagccttactgtgccctccacatcaccaccctccagctcgggcctgagccgagacgccacggccacccctccctcctccccatctatgagcagcgccctggccatcgtggggagccctaatagcccatatggggacgtgattggcctccaggtggactactggctgggccaccccggggagcggaggagggaaggcgacaagagggacgccagctcgaagaacaccctcaagagtgtcttccgctcagtgcaggtgtcccgcctgccccatagtggggaggcccagctttctggcaccatggccatgactgtggtcaccaaagaaaagaacaagaaagttcccaccatcttcctgagcaagaaaccccgagaaaaggaggtggattctaagagccaggtcattgaaggcatcagccgcctcatctgctcagccaagcagcagcagactatgctgagagtgtccatcgatggggtcgagtggagtgacatcaagttcttccagctggcagcccagtggcccacccatgtcaagcactttccagtgggactcttcagtggcagcaaggccacctgaggccctgtctcccagccactttccctcctggcactgccaccagcctcaccgcctgcgggcagggggaggccagcaggcccgggcccagcaccccttccctggcaccagggtctgcctctcactcgcccaggtcccgaaggacactgccacagggacgccttccctcccctcccctccagcccacccctgcacagcccctcctccttcccgcttttccccttctccctcctgctccaggcccaaggcgtgttggttttgccttctggtgcccatagtcccctggactgagtcccccaggccttccttcacccgacttccaaactcttccttgtggtatcagtttccttctcggaaatgagaaagctggaatcctggtccccagcaggagagcctagtcctcccccagcccctccagccaccagggtgtcctctaggatgcagctgccagatccactcactctgctgcctccagcaggacccaaggccactttcaactcttatggggttctccacctgccccagagcttctcaagggagggtaagggggcaccctgagcccacaggacccctacttcacagctcacaggggcaggaggcagctcccctgcctccaggaccctgttgctatggtgacacagcgtttctaggacagaggggcctcccagtctccccccaccacccgtgcacgacttcctcaccacccccaggttccctgcagatgtcgtgtgtgtcctgagtgtttctttggttctttgcacgccaagtctcttggttgtaccatgtgacacaccctgtgcactggtcgctgtcttcgtggcttccacccttgttaatgatgctcctgcctctgcctcccagcccctcacccagcacagctctgcctggacttggagagatgggaggcagacccccaccaccatacatgctgtctgtggcccctcagacattctgtttcatctcccattcatctccctcctcccaccgtgtcagtttttctgcctttccctgctctgttcttccccctccttaggccccagcctgggcccagacccatcctcccagccaggtttccctccagcaggctccttccctccctgtcacctccctctcaccaacccggggtctgagcccctcattcctgaccgtccgtgttctcaggagtggttgaggacacagggccccagcccagccctctgcaccccccagcccggccatctgcgccccacagcccctttggagcttttctcttgtcctctcactccttcccagaagtttttgcacagaacttcattttgaaagtgtttttctcattctccatacctcccccaagctctcctccagcccttcccagggctcagccctgctgtcctgagcgtctcctgggccagagagaggagatgggggtgggagggactgagttgatgttgggtttttcattcaataaattggtgatttcttaccgactgcaaaaaaaaaaaaaaaaaa

SEQ ID NO: 2 - protein, 963 aa, Homo sapiens
MAERGGAGGGPGGAGGGSGQRGSGVAQSPQQPPPQQQQQQPPQQPTPPKLAQATSSSSSTSAAAASSSSSSTSTSMAVAVASGSAPPGGPGPGRTPAPVQMNLYATWEVDRSSSSCVPRLFSLTLKKLVMLKEMDKDLNSVVIAVKLQGSKRILRSNEIVLPASGLVETELQLTFSLQYPHFLKRDANKLQIMLQRRKRYKNRTILGYKTLAVGLINMAEVMQHPNEGALVLGLHSNVKDVSVPVAEIKIYSLSSQPIDHEGIKSKLSDRSPDIDNYSEEEEESFSSEQEGSDDPLHGQDLFYEDEDLRKVKKTRRKLTSTSAITRQPNIKQKFVALLKRFKVSDEVGFGLEHVSREQIREVEEDLDELYDSLEMYNPSDSGPEMEETESILSTPKPKLKPFFEGMSQSSSQTEIGSLNSKGSLGKDTTSPMELAALEKIKSTWIKNQDDSLTETDTLEITDQDMFGDASTSLVVPEKVKTPMKSSKTDLQGSASPSKVEGVHTPRQKRSTPLKERQLSKPLSERTNSSDSERSPDLGHSTQIPRKVVYDQLNQILVSDAALPENVILVNTTDWQGQYVAELLQDQRKPVVCTCSTVEVQAVLSALLTRIQRYCNCNSSMPRPVKVAAVGGQSYLSSILRFFVKSLANKTSDWLGYMRFLIIPLGSHPVAKYLGSVDSKYSSSFLDSGWRDLFSRSEPPVSEQLDVAGRVMQYVNGAATTHQLPVAEAMLTCRHKFPDEDSYQKFIPFIGVVKVGLVEDSPSTAGDGDDSPVVSLTVPSTSPPSSSGLSRDATATPPSSPSMSSALAIVGSPNSPYGDVIGLQVDYWLGHPGERRREGDKRDASSKNTLKSVFRSVQVSRLPHSGEAQLSGTMAMTVVTKEKNKKVPTIFLSKKPREKEVDSKSQVIEGISRLICSAKQQQTMLRVSIDGVEWSDIKFFQLAAQWPTHVKHFPVGLFSGSKAT

SEQ ID NO: 3 - DNA, 3173 bp, Homo sapiens
ggtggacccccacgactctcccggcccttgcccgcggctcccggggggcggggcggggcgccccgggcggggtctgtgcgcaggcgcgtgagtgcgcgctctcgcgcaccggcgggcggggacgccccgtgaggcgccgtcggaggaagcgcgcgcgcacctcacttccggcgcgcgctgcgccggcggcgatttggacccgaggcggcgagctggcgccccgcccagccaatcggcggcgcggcgcgggtcggagggcgccgggcgcgcgcggggcggccgggggcgcgcggggcgcgtgcggggcgccgggcggggcggggcggacggccgcagctcgtcgccgcccgcgggcctgtccgacgccggggcccggcccgtcccctccgccgcccggcagccatgtgaccgcgccgccgccctccgcgcgcccggcccgcccgccgcgcgtccgcggcccggccgcagccccaggccgccgagggagcggcggggccggcgccatggccgagcgaggccgcctcggcctccccggcgcgctcggcgcgctcaacacgcccgtgcccatgaacctgttcgccacctgggaggtggacggctccagccccagctgcgtgcccaggttgtgcagcctgactctgaagaagctggtggtcttcaaggagctggagaaggagctgatctccgtggtgatcgctgtcaagatgcagggctccaaacgaatcctgcggtcccatgagattgtgctgccccccagtggacaagtggagacagacctggccctgaccttctccttgcagtatcctcacttcttgaagagggaaggcaacaagcttcagatcatgctgcagcgcagaaagcgctacaagaacagaaccatcctgggctacaagacgctggccgcgggctccatcagcatggctgaggtgatgcaacacccgtctgaaggtggccaggtgctgagcctctgcagcagcatcaaggaggcccccgtcaaggcggccgagatctggatcgcctccctgtccagccagcccattgaccacgaagacagcaccatgcaggccggccccaaggccaagtccacggataactactccgaggaggagtatgagagcttctcctccgagcaggaggccagtgacgacgccgtgcaggggcaggacttggacgaggacgactttgacgtggggaagccgaagaagcagcggagatcgattgtaagaacgacgtccatgaccaggcaacagaacttcaagcagaaagtggtagcgctgctgcggaggttcaaagtgtccgacgaggtcctggactcggagcaggaccctgcggagcacatccccgaggcagaggaggacctggacctcctgtatgacaccctggacatggagcaccccagcgacagcggccccgacatggaggatgacgacagcgtcctcagcacccccaagccgaagctgcggccatactttgaaggcctgtcgcactcgagctcgcagacggagattgggagcatccacagcgcccgcagccacaaggagcccccaagcccggctgacgtgcccgagaagacgcggtccctgggaggcaggcagccgagcgacagtgtctctgacacggtggccctcggtgtgccaggcccgagggagcaccctggacagcctgaggacagccccgaggctgaggcctccaccctggatgtgttcacggagaggctgccgcccagcgggaggatcaccaagacagagtcccttgtcatcccctccaccaggtccgaagggaagcaggctggccgacggggccggagcacatccttgaaggagcggcaggcagcacggccccagaatgagcgggccaacagcctggacaacgagcgctgcccggacgcccggagccagctacagatccccaggaagactgtgtatgaccagctcaaccacatcctcatctccgatgaccagcttcccgaaaacatcatccttgtcaacacctccgactggcaggggcagttcctctccgacgtcctgcagaggcacacgctccccgtggtgtgcacgtgctctcctgcggacgtccaggcggccttcagcaccatcgtctcacggatacagagatactgcaactgcaattcccagcccccgacccccgtgaagatcgccgtggcgggagcgcagcattacctcagtgccatcctgcggctctttgtggagcagctgtcccacaagacacccgactggctcggctacatgcgcttcctggtcatcccactgggctcccaccccgtggccaggtacctaggctccgtggactaccgctacaacaacttcttccaggacctggcctggagagacctgttcaacaagctggaggcccagagtgcggtacaggacacgccagacattgtgtcacgcatcacgcagtacatcgcaggggccaactgtgcccaccagctccccatcgcagaggccatgctgacctacaagcagaagagccctgacgaagagtcctcccaaaagttcattccctttgtcggggttgtgaaggttggaattgtggagccatcttcggccacatcaggcgactcggacgacgcggccccctcgggctctggcacgctctcatccaccccgccgtccgcatctcctgcggccaaggaggcctcacccaccccgccctcctccccgtcggtgagcggaggcctgtcctcccccagccagggtgtcggcgccgagctgatggggctgcaggtggactactggacggcagcacagcctgcggacaggaagagggacgccgagaagaaggacctgcctgtcaccaaaaacacgctcaagtgcactttccggtccctccaggtcagcaggctgcccagcagcggcgaggctgcagccacgcccaccatgtccatgaccgtggtcaccaaggagaagaacaagaaggtgatgtttctgcccaagaaagcgaaggacaaggacgtggagtctaagagccagtgcattgagggcatcagccggctcatctgcactgccaggcagcagcagaacatgctgcgggtcctcatcgacggcgtggagtgcagcgacgtcaagttcttccagctggccgcgcagtggtcctcgcacgtgaagcacttccccatctgcatcttcggacactccaaggccaccttctag

SEQ ID NO: 4 - protein, 889 aa, Homo sapiens [sequence omitted]